SEQUENCE LISTING (8 sequences)

SEQ ID NO: 1 - protein, 11 aa, synthetic construct
  source 1..11: note = light chain CDR sequence CDR1
KASQDVSIAVA

SEQ ID NO: 2 - protein, 7 aa, synthetic construct
  source 1..7: note = light chain CDR sequence CDR2
SASYRYT

SEQ ID NO: 3 - protein, 9 aa, synthetic construct
  source 1..9: note = light chain CDR sequence CDR3
QQHYITPLT

SEQ ID NO: 4 - protein, 5 aa, synthetic construct
  source 1..5: note = heavy chain CDR sequence CDR1
NYGMN

SEQ ID NO: 5 - protein, 17 aa, synthetic construct
  source 1..17: note = heavy chain CDR sequence CDR2
WINTYTGEPTYTDDFKG

SEQ ID NO: 6 - protein, 12 aa, synthetic construct
  source 1..12: note = heavy chain CDR sequence CDR3
GGFGSSYWYFDV

SEQ ID NO: 7 - protein, 107 aa, synthetic construct
  source 1..107: note = Sacituzumab VL
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 8 - protein, 121 aa, synthetic construct
  source 1..121: note = Sacituzumab VH
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

(19) United States
(12) Patent Application Publication
(10) Pub. No.: US 2025/0152731 A1
(43) Pub. Date: May 15, 2025

(54) ANTI-TROP2 ANTIBODY COMBINATION THERAPIES AND METHODS OF USE THEREOF

(71) Applicant: BOARD OF REGENTS, THE UNIVERSITY OF TEXAS SYSTEM, Austin, TX (US)

(72) Inventors: Funda MERIC-BERNSTAM, Houston, TX (US); Ming ZHAO, Houston, TX (US)

(21) Appl. No.: 18/698,696

(22) PCT Filed: Oct. 10, 2022
(86) PCT No.: PCT/US2022/077860

§ 371 Date: Apr. 4, 2024

Related U.S. Application Data
(60) Provisional application No. 63/254,005, filed on Oct. 8, 2021.

(51) Int. Cl. A61K 47/68; A61K 45/06; A61P 35/00; C07K 16/30

(52) U.S. Cl. CPC A61K 47/6855; A61K 45/06; A61K 47/68037; A61P 35/00; C07K 16/30

(57) ABSTRACT

According to various aspects of this disclosure, the present disclosure relates to a method of treating cancer in a subject in need thereof comprising administering to the subject an anti-TROP2 antibody drug conjugate and a therapeutic agent that increases TROP2 expression. In some aspects, the therapeutic agent that increases TROP2 expression is a DNA methyltransferase inhibitor or a Zinc Finger E-Box Binding Homeobox 1 (Zeb1) inhibitor.

### CROSS-REFERENCE TO RELATED APPLICATIONS

The present application claims the priority benefit of U.S. Provisional Patent Application No. 63/254,005, filed Oct. 8, 2021, which is incorporated herein by reference in its entirety.

### REFERENCE TO SEQUENCE LISTING SUBMITTED ELECTRONICALLY

The content of the electronically submitted sequence listing (Name: 4443_009PC01_Seqlisting_ST26.xml; Size: 9,802 bytes; and Date of Creation: Oct. 5, 2022), filed with the application, is incorporated herein by reference in its entirety.

### BACKGROUND

Tumor-associated calcium signal transducer 2 (Trop-2 or TROP2), also known as epithelial glycoprotein-1 antigen (EGP-1), is a protein that in humans is encoded by the TACSTD2 gene. Trop-2 is overexpressed in many malignant tumors.

TROP2 is being evaluated as a therapeutic target for several TROP-targeted agents, especially antibody drug conjugates (ADCs) such as sacituzumab govitecan (Gilead/Immunomedics), datopotamab deruxtecan (Dato-DXd; Astrazeneca/Daichii) and SKB264 (Klus Pharma). Sacituzumab govitecan is an antibody drug conjugate in which a Trop-2 antibody (sacituzumab) is conjugated with a topoisomerase inhibitor (SN-38). Sacituzumab govitecan is sold under the brand name Trodelvy. The therapeutic efficacy of sacituzumab govitecan on various tumor types has been evaluated in clinical studies. As a single agent, sacituzumab govitecan has been assessed in human trials on advanced, metastatic or recurrent cancer, including triple negative breast cancer (NCT04230109), HER2− breast cancer (NCT04647916, NCT04559230), urothelial cancer (NCT04527991), prostate cancer (NCT03725761), endometrial cancer (NCT04251416), Glioblastoma (NCT03995706, NCT04559230), and other solid tumors (NCT04319198, NCT03964727). FDA has approved sacituzumab govitecan for treatment of advanced urothelial cancer and metastatic triple negative breast cancer.

In recent years, sacituzumab govitecan has been also tested in combination with other therapeutic agents for certain tumors. These other therapeutic agents are directed to other pathways or mechanisms of action. These partner agents include pembrolizumab (PD-1 Antibody) for metastatic breast cancer (NCT0444886), metastatic urothelial cancer (NCT03547973), and triple negative breast cancer NCT04468061); avelumab (PD-L1 antibody) for triple negative breast cancer (NCT03971409); enfortumab vedotin (nectin 4 antibody drug conjugate) for urothelial cancer (NCT04724018); Ipilimumab (CTLA4 antibody); nivolumab (PD-1 antibody) for urothelial cancer (NCT04863885); talazoparib (PARPi) for triple negative breast cancer (NCT04039230); and berzosertib (ATRi) for small cell lung cancer (NCT04826341).

### DESCRIPTION OF FIGURES

FIG. 1A shows immunoblotting of ZEB1 knockdown. Breast cancer BCX-010CL cells were transduced with lentivirus of ZEB1 shRNA or control shRNA, followed by puromycin selection. Cell lysates were subjected to SDS-PAGE and immunoblotted with antibodies against ZEB1, E-cad, SLFN11, and TROP2.

FIG. 1B shows immunoblotting of TROP2 protein levels in the presence or absence of decitabine in BCX-010 cells (untransfected, transfected with control shRNA, or transfected with Zeb1 shRNA), BCX-011 cells, and Sum-159 cells. β-actin was used as a loading control. Cells were treated for three days.

FIG. 1C shows immunoblotting of TROP2 and E-cadherin protein levels in BCX-010 cells transfected with control shRNA or Zeb1 shRNA. Cells were treated with decitabine, panobinostat (an HDAC inhibitor), and sacituzumab govitecan at the indicated doses for three days. β-actin was used as a loading control.

FIG. 1D shows TROP2 expression and E-cadherin expression after treatment with a panel of DNA methyltransferase (DNMT) inhibitors, histone deacetylase (HDAC) inhibitors, and EZH2 inhibitors in 3 breast cancer cell lines.

FIG. 2A shows a quantitative real time PCR (qPCR) assay of TROP2 mRNA levels in BCX-010 cells transfected with control shRNA or Zeb1 shRNA. Cells were treated with decitabine and panobinostat at the indicated doses for three days. Two levels were normalized with GAPDH.

FIG. 2B shows a qPCR assay showing TROP2 mRNA levels in Sum-159 cells treated with DMSO or decitabine. TROP2 levels were normalized with GAPDH.

FIG. 2C shows a qPCR assay showing TROP2 mRNA levels in BCX-010CL cells treated with shRNA control or ZEB1 shRNA.

FIG. 2D shows immunocytochemistry of TROP2. SUM159 cells were treated with decitabine at 1 μM for 3 days. After cell fixation, cell pellet blocks were processed and probed with anti-TROP2 antibody, followed by hematoxylin staining.

FIG. 3A shows methylation-specific PCR (MSP). Genomic DNA was extracted from BCX-010CL and BCX-011CL cells treated decitabine at 1 μM for 3 days, or from ZEB1 knockdown cells, and then subjected bisulfite CT conversion, followed by MSP using methylation specific primers specific to a CpG region in the TROP2 promoter. GAPDH was used as a control.

FIG. 3B shows immunoblotting of DNA methyltransferases (DNMTs). Breast cancer cell lines were treated with decitabine at 1 μM for 3 days. DNMT isoforms were detected by immunoblotting with antibodies against DNMT1, DNMT3A, and DNMT3B.

FIG. 4A shows the combination of decitabine and/or sacituzumab govitecan on BCX-010 cells transfected with control shRNA or Zeb1 shRNA. Cells were treated with decitabine and/or sacituzumab govitecan at six diluted dose concentrations ranging from 1-100,000 nM (decitabine) and 0.05-5000 nM (sacituzumab govitecan). After four days, cell viability was measured using sulforhodamine B (SRB) staining. Drug IC50s and combination index (CI) were determined using the CalcuSyn software (CI<1.0: synergistic; CI=1.0: additive; CI>1.0: antagonistic).

FIGS. 4B-4D shows the IC50 for BCX-010 (FIG. 4B), BCX-011 (FIG. 4C), and Sum-159 (FIG. 4D) cells in the presence of decitabine and/or sacituzumab govitecan. Cells were treated with decitabine and/or sacituzumab govitecan at six diluted dose concentrations ranging from 1-100,000 nM (decitabine) and 0.05-5000 nM (sacituzumab govitecan). After four days, cell viability was measured using SRB staining. Drug IC50s was determined using the CalcuSyn software.

FIG. 5A shows an Annexin V flow cytometry analysis that measures apoptosis in BCX-010 cells that were transfected with control shRNA or Zeb1 shRNA. The BCX-010 cells were treated with decitabine (1 μM) and/or sacituzumab govitecan (0.1 μM) for three days. After the three days, cells were stained with Annexin V. Flow cytometry was performed to sort Annexin V positive cells (apoptotic cells).

FIGS. 5B-5D shows an Annexin V flow cytometry analysis that measures apoptosis in BCX-010 (FIG. 5B), BCX-011 (FIG. 5C), and Sum-159 cells (FIG. 5D). The cells were treated with decitabine (1 μM) and/or sacituzumab govitecan (0.1 μM) for three days. After the three days, cells were stained with Annexin V. Flow cytometry was performed to sort Annexin V positive cells (apoptotic cells).

FIGS. 6A-6E show a colony assay of BCX-010 (FIGS. 6A-6B), BCX-011 (FIG. 6C-6D), and Sum-159 cells (FIG. 6E-6H) after treatment with vehicle, sacituzumab govitecan (200 nM), and/or decitabine (200 nM) for 10 days. FIGS. 6A-6C show agar plates after 10 days of colony formation. FIG. 6G shows total colony area as measured by the ImageJ software. FIG. 6H shows the total colony count as measured by the ImageJ software.

FIGS. 7A-7B show a colony assay of BCX-010 cells transfected with control shRNA (FIG. 7A) or Zeb1 shRNA (FIG. 7B) after treatment with vehicle, sacituzumab govitecan (20 nM), and/or decitabine (5 nM) for 14 days. After 14 days, cell colonies formed on the plates, were fixed with 10% formalin, and stained with 0.05% crystal violet. FIG. 7C shows a cell survival assay in which shRNA control and ZEB1 shRNA cell lines were treated with decitabine, sacituzumab govitecan or their combination at concentrations in serial dilutions for 3 days. FIG. 7D shows an apoptosis assay in which cells were treated with decitabine at 1 μM, SG at 0.1 μM, or their combination for 3 days. Annexin V positive apoptotic cells were measured by flow cytometry. Percentage of apoptotic cells over total cell populations was calculated.

FIG. 8A shows immunoblotting to detect TROP2 expression. BCX-010CL cells were transfected with TROP2 expression plasmid or empty vector. After G-418 selection, immunoblotting was performed to detect TROP2 expression.

FIG. 8B shows the IC50 (nM) levels of Sacituzumab govitecan in BCX-010CL and BCX-011CL cells in a cell survival assay. TROP2-overexpressing cells and control cells were treated with SG at concentrations in serial dilutions for 3 days. SG IC50 was calculated.

FIG. 9 shows immunoblotting for signaling activity in DDR and apoptotic pathways in breast cancer cell lines. BCX-010CL (left panel) and SUM159 (right panel) cells were treated with decitabine at 1 μM, sacituzumab govitecan at 0.1 μM, and their combination for 1, 2, 3 days. Cell lysates were subjected to SDS-PAGE, followed by immunoblotting using antibodies against cleaved PARP1, cleaved Caspase 3, TROP2, SLFN11, and RAD51, and also against phospho-KAP1, γH2AX, phospho-CHK1, phospho-CHK2, and their total proteins. B-actin served as protein loading control.

FIG. 10A shows immunoblotting of SLFN11 expression in breast cancer cell lines. Cell lines BCX-010CL, HCC-1937, HCC-1143, and MDA-MB-157 were treated with decitabine at 1 μM for 3 days. SLFN11 and TROP2 proteins in cell lysates were detected by immunoblotting.

FIG. 10B shows the IC50 (nM) of SN-38 in BCX-010CL cells in a cell survival assay. BCX-010CL cells were treated with SN-38 at serial dilutions, with or without decitabine co-treatment for 3 days. IC50s of individual agents and combination index were calculated.

FIG. 10C shows the IC50 of Sacituzumab govitecan in breast cancer cell lines in a cell survival assay. BCX-010CL, HCC-1954, HCC-1143, and MDA-MB-157 cells were treated with Sacituzumab govitecan at serial dilutions, with or without decitabine co-treatment for 3 days. IC50s of individual agents and combination index were calculated.

FIG. 10D shows immunoblotting of SLFN11 and TROP2 expression in a panel of 48 cell lines including 27 breast cancer cell lines. SLFN11 and TROP2 proteins in cell lysates were detected by immunoblotting.

FIG. 10E shows immunoblotting of topoisomerase I (TOP1) expression in breast cancer cell lines based on increasing concentrations of TOP1 inhibitor SN-38, TOP1 inhibitor Dxd, Decitabine, Decitabine+SN-38, and Decitabine+Dxd.

FIG. 11A shows an Epithelial-mesenchymal transition assays on three BCX-010 cell lines (parental, transfected with control shRNA, and transfected with Zeb1 shRNA). Migration assays, invasion assays, and a soft agar assay (analyzing anchorage-independent growth) were performed.

FIGS. 11B-11C shows an Epithelial-mesenchymal transition assays on BCX-010 cell lines transfected with TROP2 or control. Migration assays (FIG. 11B) and a soft agar assay (analyzing anchorage-independent growth) (FIG. 11C) were performed.

FIG. 12A shows sacituzumab govitecan-Alexa-488 binding to cell membrane of TROP2 overexpressing cells.

FIG. 12B shows sacituzumab govitecan-Alexa-488 internalization in MDA-MB-468 cells.

FIG. 13 shows the mechanism of decitabine action. In tumor cells, decitabine decreases promoter methylation of the TACSTD2 gene by inhibiting DNA methyltransferase 1 (DNMT1), activating expression of TACSTD2/TROP2. On cell membrane, TROP2 protein binds to TROP2 ADC drug sacituzumab govitecan (SG). After internalization, SG is proteolytically cleaved in lysosome to release payload SN-38. SN-38 then moves into nucleus where it inhibits topoisomerase 1 (TOP1), leading to DNA damage, cell cycle arrest, and cell death. Decitabine also increases SLFN11 expression which further strengthens the chemocytoxic effect of SN-38 in the tumor cells.

### BRIEF SUMMARY

The present disclosure provides methods and combination therapies to treat various cancers, particularly cancers with low Trop-2 expression. The combination therapy can include an anti-TROP2 antibody drug conjugate (e.g., sacituzumab govitecan) and an agent that increases expression of TROP2.

In some aspects, the present disclosure provides a method of treating a tumor or a cancer in a subject in need thereof comprising administering to the subject an anti-TROP2 antibody drug conjugate (ADC) and a therapeutic agent that increases TROP2 expression.

In some aspects, the therapeutic agent comprises a DNA methyltransferase inhibitor.

In some aspects, the DNA methyltransferase inhibitor is decitabine.

In some aspects, the therapeutic agent comprises a Zinc Finger E-Box Binding Homeobox 1 (Zeb1) inhibitor.

In some aspects, the present disclosure provides a method of treating a tumor or a cancer in a subject in need thereof comprising administering to the subject an anti-TROP2 antibody drug conjugate (ADC) and a therapeutic agent comprising a DNA methyltransferase inhibitor and/or a Zinc Finger E-Box Binding Homeobox 1 (Zeb1) inhibitor.

In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a shRNA targeting Zeb1.

In some aspects, the anti-TROP2 antibody drug conjugate comprises a cytotoxic drug selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor and a pro-apoptotic agent.

In some aspects, the anti-TROP2 antibody drug conjugate comprises a cytotoxic drug selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the anti-TROP2 antibody drug conjugate comprises a cytotoxic drug selected from the group consisting of SN-38, pro-2-pyrrolinodoxorubicin (pro-2-PDox), paclitaxel, calichemicin, DM1, DM3, DM4, MMAE, MMAD, MMAF, and deruxtecan.

In some aspects, the anti-TROP2 antibody drug conjugate comprises a SN-38 cytotoxic drug.

In some aspects, the anti-TROP2 antibody drug conjugate comprises sacituzumab or a functional fragment thereof. In some aspects, the anti-TROP2 antibody drug conjugate comprises datopotamab or a functional fragment thereof.

In some aspects, the anti-TROP2 antibody drug conjugate is sacituzumab govitecan. In some aspects, the anti-TROP2 antibody drug conjugate is datopotamab deruxtecan. In some aspects, the anti-TROP2 antibody drug conjugate is SKB264.

In some aspects, the cancer is selected from the group consisting of brain cancer, breast cancer, cervical cancer, colorectal cancer, esophageal cancer, lung cancer, lymphomas, ovarian cancer, pancreatic cancer, prostate cancer, stomach cancer, thyroid cancer, bladder cancer, uterine cancer, and a carcinoma.

In some aspects, the breast cancer is triple negative breast cancer.

In some aspects, the breast cancer is HER2 negative breast cancer.

In some aspects, the bladder cancer is urothelial cancer.

In some aspects, the uterine cancer is endometrial cancer.

In some aspects, the brain cancer is glioblastoma multiforme.

In some aspects, the lung cancer is small cell lung cancer.

In some aspects, the therapeutic agent is administered prior to administration of the anti-TROP2 antibody drug conjugate.

In some aspects, the therapeutic agent and the anti-TROP2 antibody drug conjugate are administered simultaneously or sequentially.

In some aspects, the therapeutic agent and the anti-TROP2 antibody drug conjugate are administered in the same composition.

In some aspects, the therapeutic agent that increases TROP2 expression and the anti-TROP2 antibody drug conjugate are administered in different compositions.

In some aspects, the therapeutic agent and the anti-TROP2 antibody drug conjugate are administered intratumorally and/or intravenously.

In some aspects, the present disclosure provides a method of treating a subject in need thereof comprising determining TROP2 expression level in a tumor sample from the subject and administering to the subject having a low expression level of TROP2 in the tumor relative to a reference sample an anti-TROP2 antibody drug conjugate and a therapeutic agent that increases expression of TROP2.

In some aspects, the tumor sample is obtained by biopsy.

In some aspects, the tumor sample is a tissue sample.

In some aspects, expression of TROP2 is measured by protein expression of TROP2. In some aspects, the protein expression of TROP2 is measured by immunohistochemistry that indicates the histology of the tumor sample. In some aspects, the histology of the tumor sample does not indicate high levels of TROP2.

In some aspects, expression of TROP2 is measured by mRNA expression of TROP2.

In some aspects, the therapeutic agent comprises a DNA methyltransferase inhibitor.

In some aspects, the DNA methyltransferase inhibitor is decitabine.

In some aspects, the therapeutic agent comprises a Zeb1 inhibitor.

In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a shRNA targeting Zeb1.

In some aspects, the anti-TROP2 antibody drug conjugate comprises a cytotoxic drug selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor, and a pro-apoptotic agent.

In some aspects, the anti-TROP2 antibody drug conjugate comprises a cytotoxic drug selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the anti-TROP2 antibody drug conjugate comprises a cytotoxic drug selected from the group consisting of SN-38, pro-2-pyrrolinodoxorubicin (pro-2-PDox), paclitaxel, calichemicin, DM1, DM3, DM4, MMAE, MMAD, MMAF, and deruxtecan.

In some aspects, the anti-TROP2 antibody drug conjugate comprises sacituzumab or a functional fragment thereof. In some aspects, the anti-TROP2 antibody drug conjugate comprises datopotamab or a functional fragment thereof.

In some aspects, the anti-TROP2 antibody drug conjugate is sacituzumab govitecan. In some aspects, the anti-TROP2 antibody drug conjugate is datopotamab deruxtecan. In some aspects, the anti-TROP2 antibody drug conjugate is SKB264.

In some aspects, the cancer is selected from the group consisting of brain cancer, breast cancer, cervical cancer, colorectal cancer, esophageal cancer, lung cancer, lymphomas, ovarian cancer, pancreatic cancer, prostate cancer, stomach cancer, thyroid cancer, bladder cancer, uterine cancer, and a carcinoma.

In some aspects, the breast cancer is triple negative breast cancer.

In some aspects, the breast cancer is HER2 negative breast cancer.

In some aspects, the bladder cancer is urothelial cancer.

In some aspects, the uterine cancer is endometrial cancer.

In some aspects, the brain cancer is glioblastoma multiforme.

In some aspects, the lung cancer is small cell lung cancer.

In some aspects, the therapeutic agent that increases TROP2 expression is administered prior to administration of the anti-TROP2 antibody drug conjugate.

In some aspects, the therapeutic agent that increases TROP2 expression and the anti-TROP2 antibody drug conjugate are administered simultaneously or sequentially.

In some aspects, the therapeutic agent that increases TROP2 expression and the anti-TROP2 antibody drug conjugate are administered in the same composition.

In some aspects, the therapeutic agent that increases TROP2 expression and the anti-TROP2 antibody drug conjugate are administered in different compositions.

In some aspects, the therapeutic agent that increases TROP2 expression and the anti-TROP2 antibody drug conjugate are administered intratumorally.

In some aspects, the administration increases cancer cell death and/or reduces cancer cell growth in the subject.

In some aspects, the present disclosure provides a composition comprising an anti-TROP2 antibody drug conjugate and a therapeutic agent that increases TROP2 expression.

In some aspects, the composition further comprises at least one pharmaceutically acceptable excipient.

In some aspects, the at least one pharmaceutically acceptable excipient is a pharmaceutically acceptable carrier.

In some aspects, the therapeutic agent comprises a DNA methyltransferase inhibitor.

In some aspects, the DNA methyltransferase inhibitor is decitabine.

In some aspects, the therapeutic agent comprises a Zeb1 inhibitor.

In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a shRNA targeting Zeb1.

In some aspects, the present disclosure provides a kit for treating a subject in need thereof comprising (a) anti-TROP2 antibody drug conjugate and (b) a therapeutic agent that increases TROP2 expression.

In some aspects, the therapeutic agent comprises a DNA methyltransferase inhibitor.

In some aspects, the DNA methyltransferase inhibitor is decitabine.

In some aspects, the therapeutic agent comprises a Zeb1 inhibitor.

In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a short hairpin RNA (shRNA) targeting Zeb1.

In some aspects, the anti-TROP2 antibody drug conjugate comprises a cytotoxic drug selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor, and a pro-apoptotic agent.

In some aspects, the anti-TROP2 antibody drug conjugate comprises a cytotoxic drug selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the anti-TROP2 antibody drug conjugate comprises a cytotoxic drug selected from the group consisting of SN-38, pro-2-pyrrolinodoxorubicin (pro-2-PDox), paclitaxel, calichemicin, DM1, DM3, DM4, MMAE, MMAD, MMAF, and deruxtecan.

In some aspects, the anti-TROP2 antibody drug conjugate comprises sacituzumab. In some aspects, the anti-TROP2 antibody drug conjugate comprises datopotamab or a functional fragment thereof.

In some aspects, the anti-TROP2 antibody drug conjugate is sacituzumab govitecan. In some aspects, the anti-TROP2 antibody drug conjugate is datopotamab deruxtecan. In some aspects, the anti-TROP2 antibody drug conjugate is SKB264.

In some aspects, the anti-TROP2 antibody drug conjugate is sacituzumab govitecan and the therapeutic agent is decitabine and/or a Zeb1 inhibitor. In some aspects, the anti-TROP2 antibody drug conjugate is datopotamab deruxtecan and the therapeutic agent is decitabine and/or a Zeb1 inhibitor. In some aspects, the anti-TROP2 antibody drug conjugate is SKB264 and the therapeutic agent is decitabine and/or a Zeb1 inhibitor.

In some aspects, the present disclosure provides a combination therapy for the treatment of cancer in a subject, wherein the combination therapy comprises an anti-TROP2 antibody drug conjugate and a therapeutic agent that increases TROP2 expression.

In some aspects, the therapeutic agent comprises a DNA methyltransferase inhibitor.

In some aspects, the DNA methyltransferase inhibitor wherein the therapeutic agent is decitabine.

In some aspects, the therapeutic agent comprises a Zeb1 inhibitor.

In some aspects, the present disclosure provides a combination therapy for the treatment of cancer in a subject, wherein the combination therapy comprises an anti-TROP2 antibody drug conjugate and a therapeutic agent comprising the DNA methyltransferase inhibitor and/or a Zeb1 inhibitor.

In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a shRNA targeting Zeb1.

In some aspects, the anti-TROP2 antibody drug conjugate comprises a cytotoxic drug selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor, and a pro-apoptotic agent.

In some aspects, the anti-TROP2 antibody drug conjugate comprises a cytotoxic drug selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the anti-TROP2 antibody drug conjugate comprises a cytotoxic drug selected from the group consisting of SN-38, pro-2-pyrrolinodoxorubicin (pro-2-PDox), paclitaxel, calichemicin, DM1, DM3, DM4, MMAE, MMAD, MMAF, and deruxtecan.

In some aspects, the anti-TROP2 antibody drug conjugate comprises sacituzumab or a functional fragment thereof.

In some aspects, the anti-TROP2 antibody drug conjugate is sacituzumab govitecan.

In some aspects, the therapeutic agent and the anti-TROP2 antibody drug conjugate are administered in the same composition.

In some aspects, the therapeutic agent and the anti-TROP2 antibody drug conjugate are administered in different compositions.

In some aspects, the subject has a tumor or cancer with low TROP2 expression.

In some aspects, the composition, kit, or combination therapy disclosed herein is for use in increasing cancer cell death and/or reducing cancer cell growth.

In some aspects, the composition, kit, or combination therapy disclosed herein is for use in treating a tumor or a cancer in a subject in need thereof.

### DETAILED DESCRIPTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

It is understood that wherever aspects or embodiments are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

The terms “treatment”, “treating”, “treat” and the like are used herein to generally refer to obtaining a desired pharmacologic and/or physiologic effect. The effect may be prophylactic in terms of completely or partially preventing or reducing the risk of developing or worsening of a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease (e.g., cancer) or symptom.

The term “therapeutic agent” as used herein refers to a compound, molecule or atom that is useful in the treatment of a disease (e.g., cancer). In some aspects, a therapeutic agent can be administered separately, concurrently or sequentially with another therapeutic agent (e.g., an antibody moiety).

As used herein, the term “treating cancer” is not intended to be an absolute term. In some aspects, the compositions and methods of the invention seek to reduce the size of a tumor or number of cancer cells, cause a cancer to go into remission, or prevent growth in size or cell number of cancer cells. In some circumstances, treatment with the leads to an improved prognosis.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen.

The terms “VH” and “VH domain” are used interchangeably to refer to the heavy chain variable region of an antibody or antigen-binding fragment thereof.

The terms “VL” and “VL domain” are used interchangeably to refer to the light chain variable region of an antibody or antigen-binding fragment thereof.

As used herein, the terms “complementarity-determining region” or “CDR” are used interchangeably to refer to the antigen binding regions found within the variable region of the heavy and light chain polypeptides. Generally, antibodies or antigen binding fragments thereof comprise three CDRs in each of the VH (CDR H1 or H1; CDR H2 or H2; and CDR H3 or H3) and three in each of the VL (CDR L1 or L1; CDR L2 or L2; and CDR L3 or L3).

As used herein, the “variable regions” and “CDRs” may refer to variable regions and CDRs defined by any approach known in the art, including combinations of approaches. The identity of the amino acid residues in a particular antibody or antigen-binding fragment thereof that make up a variable region or a CDR can be determined using methods well known in the art and include methods such as sequence variability as defined by Kabat et al. (See, e g., Kabat et ah, 1992, Sequences of Proteins of Immunological Interest, 5th ed., Public Health Service, NIH, Washington D.C.), location of the structural loop regions as defined by Chothia et al. (see, e g., Chothia et ah, Nature 342:877-883, 1989), a compromise between Kabat and Chothia using Oxford Molecular's AbM antibody modeling software (now Accelrys®, see, Martin et al., 1989, Proc. Natl Acad Sci USA. 86:9268), available complex crystal structures as defined by the contact definition (see MacCallum et ah, J. Mol. Biol. 262:732-745, 1996) and the “conformational definition” (see, e.g., Makahe et ah, Journal of Biological Chemistry, 283:1 156-1 166, 2008; IMGT unique numbering for V-DOMAIN and V-LIKE-DOMAIN; lefranc numbering, Lefranc M.-P. et ah, 1997).

As used herein, the terms “constant region” and “constant domain” are interchangeable and have their meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (a), delta (d), epsilon (e), gamma (g), and mu (m), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In specific aspects, the heavy chain is a human heavy chain.

As used herein, the term “light chain” when used in reference to an antibody can refer to any distinct type, e.g., kappa (K) or lambda (l) based on the amino acid sequence of the constant domains. Light chain amino acid sequences are well known in the art. In specific aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize the specificity, affinity, and/or capability of the antibody or antigen-binding fragment thereof. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen binding fragment thereof) and its binding partner (e.g, an antigen). Unless indicated otherwise, as used herein, “binding affinity” refers to intrinsic binding affinity which reflects a 1:1 interaction between members of a binding pair (e.g, antibody or antigen binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of kₒₙ/k_{off}. kₒₙ refers to the association rate constant of, e.g., an antibody or antigen binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non contiguous regions of a polypeptide or polypeptides (conformational, non-linear, discontinuous, or non-contiguous epitope). In certain aspects, the epitope to which an antibody or antigen-binding fragment thereof binds can be determined by, e.g, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g, site-directed mutagenesis mapping). For X-ray crystallography, crystallization may be accomplished using any of the known methods in the art (e.g, Giege R et al, (1994) Acta Crystallogr D Biol Crystallogr 50(Pt 4): 339-350; McPherson A (1990) Eur J Biochem 189: 1-23; Chayen N E (1997) Structure 5:1269-1274; McPherson A (1976) J Biol Chem 251: 6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al, (2000) Acta Crystallogr D Biol Crystallogr 56(Pt 10): 1316-1323). Mutagenesis mapping studies can be accomplished using any method known to one of skill in the art. See, e.g., Champe M et al, (1995) J Biol Chem 270:1388-1394 and Cunningham B C & Wells J A (1989) Science 244: 1081-1085 for a description of mutagenesis techniques, including alanine scanning mutagenesis techniques.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain aspects, the polypeptides can occur as single chains or associated chains.

The term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. The pharmaceutical formulation can be sterile.

The terms “administer,” “administering,” “administration,” and the like, as used herein, refer to methods that may be used to enable delivery of an agent, e.g., an anti-TROP2 antibody or antigen-binding fragment thereof, to the desired site of biological action. Administration techniques that can be employed with the agents, excipients, and methods described herein are found in e.g, Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g, cow, pig, horse, cat, dog, rat, mouse, monkey or other primate, etc.). In some aspects, the subject is a human.

As used herein, the term “a subject in need of treatment” refers to an individual or subject that has been diagnosed with a disease or disorder, e.g., a cancer or a cell proliferative disorder.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat a symptom of cancer.

The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Certain aspects of the disclosure are related to a cancer therapy (e.g., a combination therapy) in which a Trop-2 targeting antibody drug conjugate (e.g., sacituzumab govitecan) is administered in combination with a therapeutic agent that increases Trop-2 expression (e.g., a DNA methyltransferase inhibitor (e.g., decitabine) and/or a Zinc Finger E-Box Binding Homeobox 1 (Zeb1) inhibitor). In some aspects, administration of decitabine and/or a Zeb1 inhibitor can be used to increase Trop-2 expression in cancer cells (e.g., cancer cells with low Trop-2 expression) to sensitize the cells to a Trop-2 antibody drug conjugate (e.g., sacituzumab govitecan) and thereby improve efficacy of the treatment. In some aspects, the combination provides a synergistic benefit (e.g., increased cancer cell death and/or reduced cancer cell growth).

### TROP2 Protein and Expression Thereof

Trophoblast cell surface antigen 2 (TROP2) is a widely expressed glycoprotein encoded by the TACSTD2 gene. TROP2 contains many aliases, such as tumor-associated calcium signal transducer 2, epithelial glycoprotein-1, M1S1, and GA733-1 (Lenárt, et al., Cancers 2020, 12, 3328; incorporated herein by reference in its entirety). TROP2 is an epithelial cell adhesion molecule (EpCAM) family member. As an intracellular calcium signal transducer, TROP2 signals cells for self-renewal, proliferation, invasion, and survival.

Human TROP2 is a 35 kDa large transmembrane protein with four N-linked glycosylation sites, consisting of 323 amino acids. The sequence of the human TROP2 protein can be found in GenBank Accession No. CAA54801.1. The extracellular domain at N-terminus starts with a 26 amino acid long hydrophobic signal peptide. The rest of the extracellular domain is 248 amino acids in length and contains 12 cysteine residues, an epidermal growth factor (EGF)-like domain, and a thyroglobulin motif. The short transmembrane domain is 23 amino acids in length, while the cytoplasmic tail is 26 amino acids in length. The mRNA and protein sequences of TROP2 are known in the art (e.g. mRNA sequence of GenBank Accession No. X13425.1, and protein sequence of GenBank Accession No. CAA54801.1).

TROP2 expression is detected in healthy epithelial cells of many organs including respiratory tract, cervix, endometrium, fallopian tubes, placenta, seminal vesicles, thymus, vagina, esophagus, skin, tonsils, cornea, breast, kidney, pancreas, prostate, salivary glands, uterus, lung, stomach, colorectum, and bile duct epithelium of the liver. TROP2 is also expressed in lungs, intestines, stomach, bladder, and kidneys during embryonal and fetal development. TROP2 protein is also detected in granule cells in all layers of the developing cerebellum, particularly in postmitotic cells, suggesting its function in regulation of cell migration. TROP2 is also re-expressed in the damaged adult stomach, and might be associated with regeneration processes.

Although TROP2 is expressed in many normal tissues, it is overexpressed in many malignant tumors, including breast, cervix, colorectal, esophagus, lung, lymphoma, ovarian, pancreatic, prostate, stomach, thyroid, urinary bladder, and uterine. TROP2 overexpression often correlates with an unfavorable prognosis and increased risk of metastasis; however, there are some cancer types in which TROP2 is downregulated (e.g., lung adenocarcinoma, squamous cell carcinoma of the esophagus) or where downregulation is correlated with poor prognosis (e.g., hepatocellular carcinoma).

In some aspects, the disclosure relates to treating tumors with low Trop-2 expression. In some aspects, TROP2 expression may be determined by measuring mRNA levels or protein levels of TROP2. In some aspects, TROP2 expression is determined by measuring mRNA levels of TROP2. In some aspects, TROP2 expression is determined by measuring protein levels of TROP2. In some aspects, the mRNA levels are measured by quantitative real time polymerase chain reaction or RNA sequencing (RNA-seq). In some aspects, the protein levels are measured by immunohistochemistry, immunocytochemistry, or western blot. In some aspects, TROP2 expression may be determined by measuring methylation of the TROP2 promoter. In some aspects, the methylation of the TROP2 promoter is measured by methylation-specific polymerase chain reaction.

### Anti-TROP2 Binding Molecules

In some aspects the compositions and methods of the disclosure comprises an anti-TROP2 antibody drug conjugate (ADC). In some aspects, the anti-TROP2 antibody drug conjugate includes at least one antibody or functional fragment thereof that binds to TROP2. In some aspects, the anti-TROP2 antibody is sacituzumab, which is also known as the humanized monoclonal antibody hRS7 (e.g., U.S. Pat. No. 7,238,785, incorporated herein by reference in its entirety). The sacituzumab antibody was generated using a murine IgG1 raised against a crude membrane preparation of a human primary squamous cell lung carcinoma. (Stein et al., Cancer Res. 50: 1330, 1990). In some aspects, the anti-TROP2 antibody comprises the light chain CDR sequences CDR1 (KASQDVSIAVA) (SEQ ID NO: 1); CDR2 (SASYRYT) (SEQ ID NO: 2); and CDR3 (QQHYITPLT) (SEQ ID NO: 3) and the heavy chain CDR sequences CDR1 (NYGMN) (SEQ ID NO: 4); CDR2 (WINTYTGEPTYTDDFKG) (SEQ ID NO: 5) and CDR3 (GGFGSSYWYFDV) (SEQ ID NO: 6). In some aspects, the anti-TROP2 antibody comprises the light chain variable region (VL) and the heavy chain variable region (VH) as displayed in Table 1.

TABLE 1

| Description | Sequence |
|---|---|
| Sacituzumab | DIQLTQSPSSLSASVGDRVSITC |
| VL | KASQDVSIAVAWYQQKPGKAPKL |
| | LIYSASYRYTGVPDRFSGSGSGT |
| | DFTLTISSLQPEDFAVYYCQQHY |
| | ITPLTFGAGTKVEIK |
| | (SEQ ID NO: 7) |
| Sacituzumab | QVQLQQSGSELKKPGASVKVSCK |
| VH | ASGYTFTNYGMNWVKQAPGQGLK |
| | WMGWINTYTGEPTYTDDFKGRFA |
| | FSLDTSVSTAYLQISSLKADDTA |
| | VYFCARGGFGSSYWYFDVWGQGS |
| | LVTVSS |
| | (SEQ ID NO: 8) |

Other anti-TROP2 antibodies that are known can be utilized in the subject ADCs. While humanized or human antibodies are preferred for reduced immunogenicity, in some aspects a chimeric antibody can be of use. Methods of antibody humanization are well known in the art and may be utilized to prepare known murine or chimeric antibody into a humanized form.

In some aspects, the anti-TROP2 can comprise a commercially available antibody or antibody having the six CDRs of a commercially available antibody selected from the group consisting of LS-C126418, LS-C178765, LS-C126416, LS-C126417 (LifeSpan BioSciences, Inc., Seattle, Wash.); 10428-MM01, 10428-MM02, 10428-R001, 10428-R030 (Sino Biological Inc., Beijing, China); MR54 (eBioscience, San Diego, Calif.); sc-376181, sc-376746, Santa Cruz Biotechnology (Santa Cruz, Calif.); MM0588-49D6, (Novus Biologicals, Littleton, Colo.); ab79976, and ab89928 (ABCAM®, Cambridge, Mass.).

In some aspects, the anti-Trop-2 antibody can be selected from sacituzumab or another known anti-Trop antibody such as any of the following: U.S. Publ. No. 2013/0089872 discloses anti-TROP2 antibodies K5-70 (Accession No. FERM BP-11251), K5-107 (Accession No. FERM BP-11252), K5-116-2-1 (Accession No. FERM BP-11253), T6-16 (Accession No. FERM BP-11346), and T5-86 (Accession No. FERM BP-11254), deposited with the International Patent Organism Depositary, Tsukuba, Japan. U.S. Pat. No. 5,840,854 disclosed the anti-TROP2 monoclonal antibody BR110 (ATCC No. HB11698). U.S. Pat. No. 7,420,040 disclosed an anti-TROP2 antibody produced by hybridoma cell line AR47A6.4.2, deposited with the IDAC (International Depository Authority of Canada, Winnipeg, Canada) as accession number 141205-05. U.S. Pat. No. 7,420,041 disclosed an anti-TROP2 antibody produced by hybridoma cell line AR52A301.5, deposited with the IDAC as accession number 141205-03. U.S. Publ. No. 2013/0122020 disclosed anti-Trop-2 antibodies 3E9, 6G11, 7E6, 15E2, 18B1. Hybridomas encoding a representative antibody were deposited with the American Type Culture Collection (ATCC), Accession Nos. PTA-12871 and PTA-12872. U.S. Pat. No. 8,715,662 discloses anti-Trop-2 antibodies produced by hybridomas deposited at the AID-ICLC (Genoa, Italy) with deposit numbers PD 08019, PD 08020 and PD 08021. U.S. Patent Application Publ. No. 20120237518 discloses anti-TROP 2 antibodies 77220, KM4097 and KM4590. U.S. Pat. No. 8,309,094 (Wyeth) discloses antibodies A1 and A3, identified by sequence listing. The Examples section of each patent or patent application cited above in this paragraph is incorporated herein by reference. Non-patent publication Lipinski et al. (1981, Proc Natl. Acad Sci USA, 78:5147-50) disclosed anti-TROP2 antibodies 162-25.3 and 162-46.2.

In some aspects, the anti-TROP2 antibody binds a human TROP2 protein (see, e.g., GenBank Accession No. CAA54801.1). In some aspects, the anti-TROP2 antibody is humanized, human or chimeric antibodies. In some aspects, the anti-TROP2 antibody is sacituzumab or a functional fragment thereof.

In some aspects, the anti-TROP2 antibody is conjugated to a cytotoxic drug. In some aspects, the cytotoxic drug is selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor, and a pro-apoptotic agent.

In some aspects, the cytotoxic drug is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

In some aspects, the cytotoxic drug is selected from the group consisting of SN-38, pro-2-pyrrolinodoxorubicin (pro-2-PDox), paclitaxel, calichemicin, DM1, DM3, DM4, MMAE, MMAD, MMAF, and deruxtecan. In some aspects, the cytotoxic drug is SN-38.

In some aspects, the anti-TROP2 antibody is conjugated to SN-38. In some aspects, the anti-TROP2 antibody drug conjugate of the disclosure is sacituzumab govitecan (e.g., Trodelvy). In some aspects, the anti-TROP2 antibody drug conjugate of the disclosure is datopotamab deruxtecan. In some aspects, the anti-TROP2 antibody drug conjugate of the disclosure is SKB264 (Klus biopharma).

### Therapeutic Agents

In some aspects, disclosed herein is a therapeutic agent that increases the expression of TROP2. In some aspects, the increase of TROP2 expression in a tumor or cancer cell (e.g., a tumor or cancer cell with low TROP2 expression) sensitizes the cells to a TROP2 antibody drug conjugate (e.g., sacituzumab govitecan) and thereby improves efficacy of the treatment. In some aspects, the expression of TROP2 is increased through demethylation of the TROP2 promoter.

In some aspects, the therapeutic agent is a DNA methyltransferase inhibitor. In some aspects, the DNA methyltransferase inhibitor inhibits human DNA methyltransferase enzymes, including one or more of DNMT1, DNMT2, DNMT3a, and DNMT3b.

In some aspects, the DNA methyltransferase inhibitor is selected from the group consisting of azacitidine, decitabine, 5,6-dihydro-5-azacytidine, fazarabine, 5-fluoro-2′-deoxycitidine, zebularine, hydralizine, procaine, procainamide, epigallocatechin gallate, psammaplin A, or(S)-2-(1,3-dioxo-1,3-dihydro-isoindol-2-yl)-3-(1H-indol-3-yl)-propionic acid, or a pharmaceutically acceptable salt thereof, preferably azacitidine, decitabine, 5,6-dihydro-5-azacytidine, fazarabine, 5-fluoro-2′-deoxycitidine, or zebularine, or a pharmaceutically acceptable salt thereof, azacitidine or decitabine, or a pharmaceutically acceptable salt thereof, azacitidine, or a pharmaceutically acceptable salt thereof. In some aspects, the DNA methyltransferase inhibitor is decitabine or a pharmaceutically acceptable salt thereof.

Administration of a DNA methyltransferase inhibitor can result in hypomethylation of the TROP2 promoter, resulting in an increase TROP2 expression in cancer cells, thereby sensitizing a tumor or cancer cells to treatment with an anti-TROP2 antibody.

In some aspects, the DNA methyltransferase inhibitor is decitabine. Decitabine (5-aza-2′-deoxycitidine), is an analogue of the natural nucleoside 2′-deoxycytidine. Decitabine is a nucleic acid synthesis inhibitor. Decitabine hypomethylates DNA by inhibiting DNA methyltransferase. Decitabine has been used in the treatment of myelodysplastic syndromes (e.g., acute myeloid leukemia).

In some aspects, the therapeutic agent is a Zinc Finger E-Box Binding Homeobox 1 (Zeb1) inhibitor. Zeb1 is a zinc finger transcription factor that is known to induce epithelial-mesenchymal transition. Knockdown of Zeb1 has been shown to increase TROP2 expression.

In some aspects, the expression of Zeb1 is modulated through RNA interference, using small interfering RNAs (siRNA) or small hairpin RNAs (shRNAs). In some aspects, the present invention relates to double stranded nucleic acid molecules including small nucleic acid molecules, such as short interfering nucleic acid (siNA), short interfering RNA (siRNA), double-stranded RNA (dsRNA), micro-RNA (miRNA), and short hairpin RNA (shRNA) molecules able to mediate RNA interference (RNAi) against Zeb1 gene expression, including cocktails of such small nucleic acid molecules and suitable formulations of such small nucleic acid molecules.

RNA mediated gene silencing is based on post-transcriptional degradation of the target mRNA induced by the endonuclease Argonaute2 which is part of the RISC complex. Sequence specificity of degradation is determined by the nucleotide sequence of the specific antisense RNA strand loaded into the RISC complex.

In some aspects, the introduction into cells of a siRNA compound results in cells having a reduced level of the target mRNA and, thus, of the corresponding polypeptide and, concurrently, of the corresponding enzyme activity.

SiRNAs specific for Zeb1 can be used as modulators of Zeb1 activity, e.g., to reduce the translation of Zeb1 mRNA. In some aspects, siRNA specific for Zeb1 can be used to increase TROP2 expression in cancer cells, thereby sensitizing the cells to treatment with an anti-TROP2 antibody.

In some aspects, the disclosure provides a double stranded nucleic acid molecule, such as a siRNA molecule, where one of the strands comprises nucleotide sequence having complementarity to a predetermined Zeb1 nucleotide sequence in a target Zeb1 nucleic acid molecule, or a portion thereof.

The RNA molecule can be used modified or unmodified. An example of modification is the incorporation of tricyclo-DNA to allow improved serum stability of oligonucleotide.

In some aspects, the Zeb1 inhibitor can include a double-stranded short interfering nucleic acid molecule that down-regulates expression of a target Zeb1 gene or that directs cleavage of a target RNA, wherein said siRNA molecule comprises about 15 to about 28 base pairs, e.g., 19 base pairs. A siRNA or RNAi inhibitor of the instant disclosure can be chemically synthesized, expressed from a vector or enzymatically synthesized.

Inhibitors of Zeb1 activity can be administrated by any suitable route, both locally or systemically depending on the nature of the molecule and the expected effect. SiRNA can be administrated locally in case of double strand molecule directly in the targeted tissue, or administrated through a vector in case of shRNA.

In some aspects, RNAi is obtained using shRNA molecules. ShRNA constructs encodes a stem-loop RNA. After introduction into cells, this stem-loop RNA is processed into a double stranded RNA compound, the sequence of which corresponds to the stem of the original RNA molecule. Such double stranded RNA can be prepared according to any method known in the art including in vitro and in vivo methods as, but not limited to, described in Sadhu et al (1987), Bhattacharyya et al, (1990) or U.S. Pat. No. 5,795,715.

For in vivo administration, shRNA can be introduced into a plasmid. Plasmid-derived shRNAs present the advantage to provide the option for combination with reporter genes or selection markers, and delivery via viral or non viral vectors. The introduction of shRNA into a vector and then into cells ensure that the shRNA is continuously expressed. The vector is usually passed on to daughter cells, allowing the gene silencing to be inherited.

The route of administration of siRNA varies from local (e.g., intratumor), direct delivery to systemic intravenous administration. The advantage of local delivery is that the doses of siRNA required for efficacy are substantially low since the molecules are injected into or near the target tissue. Local administration also allows for focused delivery of siRNA. For such direct delivery, naked siRNA can be used. “Naked siRNA” refers to delivery of siRNA (unmodified or modified) in saline or other excipients such as 5% dextrose. The siRNA can also be formulated into lipids especially liposomes.

In some aspects, siRNA can be formulated with cholesterol conjugate, liposomes or polymer-based nanoparticles. Liposomes can be used to provide increased pharmacokinetics properties and/or decreased toxicity profiles. They can allow significant and repeated in vivo delivery. In some aspects, formulation with polymers such as dynamic polyconjugates—for example coupled to N-acetylglucosamine for hepatocytes targeting—and cyclodextrin-based nanoparticles allow both targeted delivery and endosomal escape mechanisms. Others polymers such as atelocollagen and chitosan can provide therapeutic effects on subcutaneous tumor xenografts as well as on bone metastases.

In some aspects, siRNA can also be directly conjugated with a molecular entity designed to help targeted delivery. Examples of conjugates include lipophilic conjugates such as cholesterol, or aptamer-based conjugates.

In some aspects, cationic peptides and proteins can be used to form complexes with the negatively charged phosphate backbone of the siRNA duplex.

In some aspects, the Zeb1 inhibitor is selected from the group consisting of a short interfering RNA (siRNA), a short hairpin RNA (shRNA), microRNA (miRNA) or an antisense nucleic acid molecule. In some aspects, the miRNA is miR-200. In some aspects, the Zeb1 inhibitor is a shRNA targeting Zeb1.

In some aspects, the disclosure is directed to a combination of therapeutic agents (e.g., a DNA methyltransferase inhibitor disclosed herein and Zeb1 inhibitor disclosed herein). In some aspects, the combination of therapeutic agents increases the expression of TROP2. In some aspects, the increase of TROP2 expression in a tumor or cancer cell (e.g., a tumor or cancer cell with low TROP2 expression) sensitizes the cells to a TROP2 antibody drug conjugate (e.g., sacituzumab govitecan) and thereby improves efficacy of the treatment. In some aspects, decitabine and a Zeb1 inhibitor are administered in combination with the anti-TROP2 antibody. In some aspects, the administration of decitabine and/or Zeb1 inhibitors can increase TROP2 expression in certain tumors and/or cancer cells.

### Methods of Treatment/Use

Certain aspects of the disclosure are directed to a method for treating cancer or a tumor in a subject in need thereof comprising administering to the subject any of the anti-TROP2 antibody drug conjugates described herein and any of the therapeutic agents that increase TROP2 expression described herein. In some aspects, the therapeutic agent is administered to increase TROP2 expression in a tumor or cancer cell, e.g., to improve the efficacy of an anti-TROP2 antibody drug conjugate therapy.

Some aspects of the disclosure are directed to a method of treating a subject in need thereof comprising administering to the subject an anti-TROP2 antibody drug conjugate disclose herein and a therapeutic agent that increases expression of TROP2 disclosed herein. In some aspects, the anti-TROP2 antibody drug conjugate is sacituzumab govitecan. In some aspects, the therapeutic agent that increases expression of TROP2 is a DNA methyltransferase inhibitor (e.g., decitabine) and/or Zeb1 inhibitor.

In some aspects, disclosed herein is a method of treating cancer tumor in a subject in need thereof comprising (i) determining TROP-2 expression level in a tumor sample from the subject and (ii) administering to the subject with low expression level of TROP2 of TROP2 in the tumor sample (e.g., relative to a reference sample) an anti-TROP2 antibody drug conjugate and a therapeutic agent that increases expression of TROP2. In some aspects, the histology of the tumor sample does not indicate high levels of TROP2. In some aspects, the tumor sample is obtained by biopsy. In some aspects, the tumor sample is a tissue sample, a blood sample or a serum sample. In some aspects, the reference sample is from a subject with TROP2 positive cancer.

In some aspects, the expression of TROP2 is measured by protein expression of TROP2. In some aspects, the protein expression of TROP2 is measured by immunohistochemistry, immunocytochemistry, or western blot. In some aspects, the immunohistochemistry indicates the histology of the tumor sample. In some aspects, the histology of the tumor sample does not indicate high levels of TROP2. In some aspects, the histology of the tumor sample indicates low levels of TROP2.

In some aspects, the expression of TROP2 is measured by mRNA levels of TROP2. In some aspects, the mRNA expression of TROP2 is measured by quantitative real time PCR or RNA-seq.

In some aspects, TROP2 level may be determined by measuring methylation of the TROP2 promoter. In some aspects, the methylation of the TROP2 promoter is measured by methylation-specific polymerase chain reaction.

In some aspects, the therapeutic agent is a DNA methyltransferase inhibitor. In some aspects, the DNA methyltransferase inhibitor is decitabine.

In some aspects, the therapeutic agent is a Zinc Finger E-Box Binding Homeobox 1 (Zeb1) inhibitor. In some aspects, the Zeb1 inhibitor is selected from the group consisting of a short interfering RNA (siRNA), a short hairpin RNA (shRNA), micro RNA (miRNA) or an antisense nucleic acid molecule. In some aspects, the Zeb1 inhibitor is a shRNA targeting Zeb1.

In some aspects, the anti-TROP2 antibody drug conjugate is sacituzumab govitecan.

In some aspects, the cancer being treated by the methods described herein is selected from the group consisting of brain cancer, breast cancer, cervical cancer, colorectal cancer, esophageal cancer, lung cancer, lymphomas, ovarian cancer, pancreatic cancer, prostate cancer, stomach cancer, thyroid cancer, bladder cancer, and uterine cancer.

In some aspects, the breast cancer is triple negative breast cancer. In some aspects, the breast cancer is HER2 negative breast cancer. In some aspects, the bladder cancer is urothelial cancer. In some aspects, the uterine cancer is endometrial cancer. In some aspects, the brain cancer is glioblastoma multiforme. In some aspects, the lung cancer is small cell lung cancer. In some aspects, the cancer is a carcinoma.

In some aspects, the therapeutic agent that increases TROP2 expression is administered prior to administration of the anti-TROP2 antibody drug conjugate.

In some aspects, the therapeutic agent that increases TROP2 expression and the anti-TROP2 antibody drug conjugate are administered simultaneously or sequentially.

In some aspects, the therapeutic agent that increases TROP2 expression and the anti-TROP2 antibody drug conjugate are administered in the same composition.

In some aspects, the therapeutic agent that increases TROP2 expression and the anti-TROP2 antibody drug conjugate are administered in different compositions.

In some aspects, the therapeutic agent that increases TROP2 expression and/or the anti-TROP2 antibody drug conjugate are administered intratumorally. In some aspects, the therapeutic agent that increases TROP2 expression and/or the anti-TROP2 antibody drug conjugate are administered intravenously. In some aspects, the therapeutic agent that increases TROP2 expression and the anti-TROP2 antibody drug conjugate are administered subcutaneously.

In some aspects, described herein is a method for treating cancer in a subject in need thereof comprising administering to the subject an anti-TROP2 antibody drug conjugate, a first therapeutic agent that increases TROP2 expression, and a second therapeutic agent that increases TROP2 expression. In some aspects, the first therapeutic agent is a DNA methyltransferase inhibitor. In some aspects, the first therapeutic agent is a Zeb1 inhibitor. In some aspects, the second therapeutic agent is a DNA methyltransferase inhibitor. In some aspects, the second therapeutic agent is a Zeb1 inhibitor. In some aspects, the DNA methyltransferase inhibitor is decitabine. In some aspects, the Zeb1 inhibitor is selected from the group consisting of a short interfering RNA (siRNA), a short hairpin RNA (shRNA), micro RNA (miRNA) or an antisense nucleic acid molecule. In some aspects, the Zeb1 inhibitor is a shRNA targeting Zeb1.

In some aspects, the first therapeutic agent that increases TROP2 expression, the second therapeutic agent that increases TROP2 expression, and the anti-TROP2 antibody drug conjugate are administered simultaneously or sequentially.

In some aspects, the first therapeutic agent that increases TROP2 expression, the second therapeutic agent that increases TROP2 expression, and the anti-TROP2 antibody drug conjugate are administered in the same composition.

In some aspects, the first therapeutic agent that increases TROP2 expression, the second therapeutic agent that increases TROP2 expression, and the anti-TROP2 antibody drug conjugate are administered in different compositions.

In some aspects, the first therapeutic agent that increases TROP2 expression, the second therapeutic agent that increases TROP2 expression, and/or the anti-TROP2 antibody drug conjugate are administered intratumorally. In some aspects, the first therapeutic agent that increases TROP2 expression, the second therapeutic agent that increases TROP2 expression, and the anti-TROP2 antibody drug conjugate are administered intravenously and/or subcutaneously.

### Compositions and Kits

Certain aspects of the disclosure are directed to a composition comprising an anti-TROP2 antibody drug conjugate and a therapeutic agent (e.g., that increases TROP2 expression). In some aspects, the anti-TROP2 antibody drug conjugate is any of the anti-TROP2 antibody drug conjugates described herein. In some aspects, the therapeutic agent that increases TROP2 expression is any of the therapeutic agents disclosed herein (e.g., a DNA methyltransferase inhibitor and/or a Zeb1 inhibitor).

In some aspects, the composition further comprises at least one pharmaceutically acceptable excipient. In some aspects, the at least one pharmaceutically acceptable excipient is a pharmaceutically acceptable carrier.

In some aspects, described herein is a kit for treating cancer in a subject in need thereof comprising (a) anti-TROP2 antibody drug conjugate and (b) a therapeutic agent that increases TROP2 expression. In some aspects, the anti-TROP2 antibody drug conjugate is any of the anti-TROP2 antibody drug conjugates described herein. In some aspects, the therapeutic agent that increases TROP2 expression is any of the therapeutic agents described herein.

In some aspects, the composition or kid comprises a therapeutic agent that is a DNA methyltransferase inhibitor. In some aspects, the DNA methyltransferase inhibitor is decitabine.

In some aspects, composition or kid comprises a therapeutic agent that is a Zeb1 inhibitor. In some aspects, the Zeb1 inhibitor is selected from the group consisting of a short interfering RNA (siRNA), a short hairpin RNA (shRNA), micro RNA (miRNA) or an antisense nucleic acid molecule. In some aspects, the Zeb1 inhibitor is a shRNA targeting Zeb1.

In some aspects, described herein is a combination therapy for the treatment of cancer in a subject, wherein the combination therapy comprises an anti-TROP2 antibody drug conjugate and a therapeutic agent that increases TROP2 expression.

In some aspects, described herein is a combination therapy for the treatment of cancer in a subject, wherein the combination therapy comprises an anti-TROP2 antibody drug conjugate and a therapeutic agent comprising a DNA methyltransferase inhibitor (e.g., decitabine) and/or a Zeb1 inhibitor.

In some aspects, the therapeutic agent is a DNA methyltransferase inhibitor. In some aspects, the DNA methyltransferase inhibitor wherein the therapeutic agent is decitabine.

In some aspects, the therapeutic agent is a Zeb1 inhibitor. In some aspects, the Zeb1 inhibitor is selected from the group consisting of a short interfering RNA (siRNA), a short hairpin RNA (shRNA), micro RNA (miRNA) or an antisense nucleic acid molecule.

In some aspects, the Zeb1 inhibitor is a shRNA targeting Zeb1.

In some aspects, the anti-TROP2 antibody drug conjugate is sacituzumab govitecan (e.g., Trodelvy).

In some aspects, the therapeutic agent that increases TROP2 expression and the anti-TROP2 antibody drug conjugate are administered in the same composition.

In some aspects, the therapeutic agent that increases TROP2 expression and the anti-TROP2 antibody drug conjugate are administered in different compositions.

In some aspects, the subject has a cancer with low TROP2 expression.

The following examples are illustrative and do not limit the scope of the claimed aspects.

### EXAMPLES

### Example 1. Decitabine Treatment and Zeb1 Knockdown Enhance TROP2 Protein Expression in Breast Cancer Cell Lines

### TROP2 Chromogenic Immunohistochemistry

To assess TROP2 protein expression in breast cancer samples, a total of 42 formalin-fixed and paraffin embedded (FFPE) sections from surgically resected specimens were stained with anti-TROP2 antibody 1:2000 (clone ERP20043, Abcam, cat #214488), using an automated immunohistochemistry system (Leica Bond Rx, Leica Biosystems). Additionally, two TMAs developed from breast cancer samples obtained by either image-guided biopsy or surgical resection were stained. TROP2 membrane expression was evaluated and the percentage staining positivity (0-100%) and staining intensity (0=no staining, 1+=weak staining, 2+=moderate staining, and 3+=strong staining) were used to generate H-scores (0-300). TROP2 expression was classified as no TROP2 expression (H-score 0), low TROP2 expression (H-score 1-100), intermediate TROP2 expression (H-score 101-200), and high TROP2 expression (H-score 201-300). Tumors with no TROP2 expression (H-score 0) were considered TROP-2 negative, and tumors with any TROP2 expression (H-score 1-300) were considered TROP2 positive.

### Cell Lines, Plasmids, Drugs and Other Reagents

Breast cancer cell lines MDA-BCX-010CL (BCX-010CL) and MDA-BCX-011CL (BCX-011CL) were generated from PDXs MDA-BCX-010 (BCX-010) and MDA-BCX-011 (BCX-011), both generated from metaplastic breast cancers. Breast cancer cell lines HCC-1937, HCC-1143, MDA-MB-157, and SUM159, were purchased from American Type Culture Collection (ATCC). Cells were cultured in Dulbecco's modified Eagle's medium/F-12 (DMEM) supplemented with 10% fetal bovine serum at 37° and humidified 5% CO2. Small hairpin RNA (shRNA) expression plasmids for Zeb1 (TRCN0000017565 and TRCN0000235850) were purchased from Sigma-Aldrich. Expression plasmid pCMV6-entry-TROP2 was purchased from Origene. DNA methyltransferase inhibitors decitabine (5-Aza-2′-deoxycytidine, DAC), azacitidine (5-Azacytidine) and GSK-3685032; HDAC inhibitors panobinostat, vorinostat, belinostat; EZH2 inhibitor tazemetostat; and TOP1 inhibitor SN-38 were purchased from MedChemExpress (MCE). Sacituzumab govitecan was obtained from MD Anderson Cancer Center pharmacy. Anti-TROP2 antibody was purchased from Abcam. Antibodies against ZEB1, E-cadherin, cleaved PARP, cleaved Caspase 3, H2AX, γH2AX (Ser139), KAP1, phospho-KAP1 (Ser824), CHK1, phospho-CHK1 (Ser345), CHK2, phospho-CHK2 (Thr68), RAD51, SLFN11, DNMT1, DNMT3A, and DNMT3B were purchased from Cell Signaling Technology (CST). Anti-β-actin antibody (#A5441) was purchased from Sigma-Aldrich. Second antibodies Goat-anti-Rabbit-Alexa Fluor-680 (#A21076) and Goat-anti-Mouse-Dylight-800 (#610145-121) were purchased from Life Tech and Rockland Immunochemicals, respectively.

ZEB1 shRNA Knockdown

HEK-293 cells were seeded in 6-well plates at 3×10⁵ cells/well overnight. Cells were transfected with viral plasmids of ZEB1 shRNAs using Lipofectamine 3000 Kit (ThermoFisher). Lentivirus media were harvested at 48 hours after transfection. BCX-010CL cells were seeded in 6-well plates at 3×10⁵ cells/well overnight. Cells were infected with Zeb1 shRNA lentivirus for 2-3 days, followed by puromycin selection for 2-3 passages. ZEB1 western blotting (WB) was performed to select knockdown cells.

### Western Blot Analysis

Following treatments, cells were washed with cold PBS and lysed in 1× Laemmli buffer. The protein concentrations in the cell lysates were measured using Pierce BCA protein assay Kit (ThermoFisher). The same amount protein for each sample (20-50 μg/lane) was loaded to SDS-PAGE gel, followed by transferring proteins to a 0.2 μm nitrocellulose membrane (Bio-Rad Laboratories). Membranes were blocked with blocking buffer Blocker Casein in PBS (ThermoFisher) at room temperature for 1 hour, followed by immunoblotting with the primary antibodies in 5% BSA TBST buffer at room temperature overnight. After washing with TBST buffer, the immunoblotting membrane was then probed with the secondary antibodies with fluorescence conjugation. The immunoblots were visualized and the immunoblotting signal intensity quantitated using the Odyssey IR imaging system (Li-Cor Biosciences).

### Xenograft Generation

Surgical samples and blood were collected from consenting patients. Tumors were first implanted into the flanks or fourth mammary fat pad of highly immunodeficient NGS mice followed by implantation in nu/nu mice for experimentation (The Jackson Laboratory, Bar Harbor ME).

### Statistical Analysis

For in vitro studies, student's t-test was performed to compare groups. Pearson test was used for correlation between groups. For in vivo studies, one-way ANOVA tests followed by Tukey was used by Dunnett's multiple comparison. Data was presented as means±SEM. Log-rank test was used for comparison of Kaplan-Meier survival curves. Figures were generated using Prism version 8.0.0 (San Diego, CA).

TROP2 protein expression was measured in response to decitabine treatment and/or Zinc Finger E-Box Binding Homeobox 1 (Zeb1) knockdown treatment in breast cancer cell lines. BCX-010 and BCX-011 cells were originally established by isolating the primary tumor cells from patient-derived xenograft (PDX) tumors of breast cancer patients. Sum-159 cells are a triple negative breast cancer cell line.

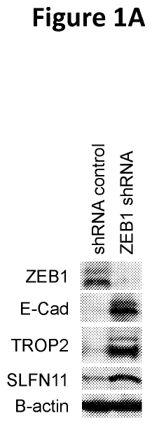

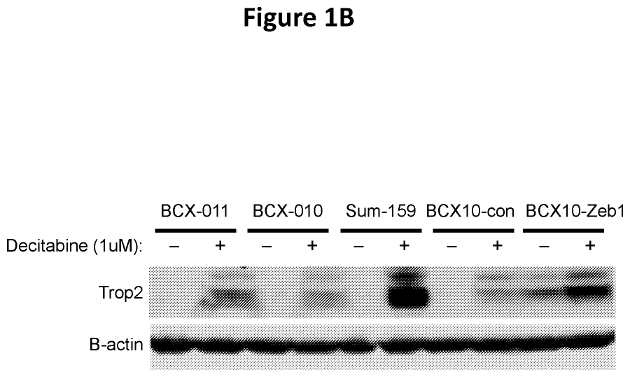

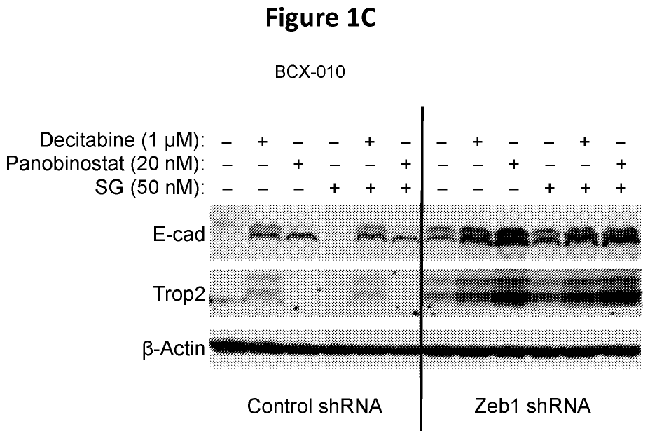

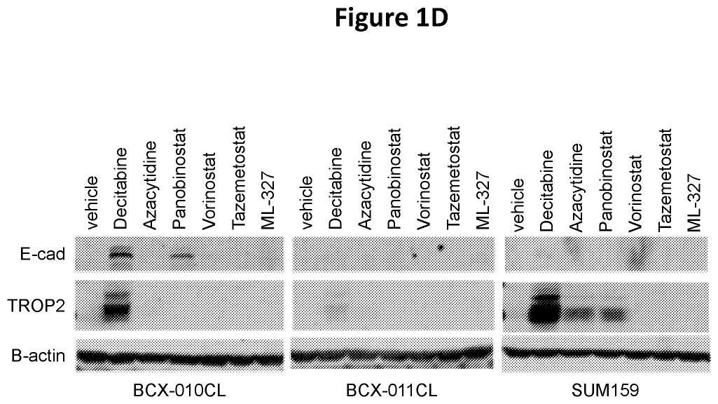

Breast cancer BCX-010CL cells were transduced with lentivirus of ZEB1 shRNA or control shRNA, followed by puromycin selection. Cell lysates were subjected to SDS-PAGE and immunoblotted with antibodies against ZEB1, E-cad, SLFN11, and TROP2 (FIG. 1A).

Five cell lines (BCX-011, BCX-010, Sum-159, BCX-010 transfected with control shRNA, and BCX-010 transfected with Zeb1 shRNA) were treated with decitabine (a DNA methyltransferase inhibitor) at 1 μM for 3 days (FIG. 1B). To knockdown Zeb1, BCX-010 cells were transduced with a lentivirus containing Zeb1 shRNA, followed by puromycin selection.

BCX-010 control and Zeb1 KD cells were also treated with 1 M decitabine, 20 nM panobinostat (an HDAC inhibitor), and/or 50 nM sacituzumab govitecan (SG, a TROP2 ADC drug) for three days (FIG. 1C).

Breast cancer cell lines BCX-010CL, BCX-011CL, and SUM159 cells were treated with decitabine, azacytidine, panobinostat, vorinostat, tazemetostat at 1, 1, 0.02, 1, 1, and 10 μM respectively for 3 days, followed by immunoblotting for E-cad and TROP2 with β-actin as control (FIG. 1D). Several DNMT and HDAC inhibitors enhanced expression of TROP2 and E-cadherin (E-cad) (FIG. 1D). Among these epigenetic modulators, the DNMT inhibitor decitabine was the most effective enhancer of TROP2 expression.

The efficiency of the drug treatments and/or Zeb1 knockdown was confirmed by western blot. The treated cells were lysed. The cell lysate samples were loaded onto SDS-PAGE gel for the western blot. Antibodies ab-214488 and cst-3195 were used to detect TROP2 and E-cadherin respectively. β-actin was used as loading control (FIG. 1C).

The immunoblot showed that decitabine increased TROP2 protein expression in all 5 cell lines (FIG. 1B). Furthermore, Zeb1 shRNA mediated knockdown increased E-cadherin, TROP2, and SLFN11 protein expression in breast cancer cell lines (FIGS. 1A, 1B and 1C). Finally, decitabine treatment and Zeb1 shRNA mediated knockdown also increased E-cadherin expression in BCX-010CL cells.

### Example 2. Decitabine Treatment and Zeb1 KD Enhance TROP2 mRNA Expression in BCX-010 and Sum-159 Cells

TROP2 mRNA expression was measured in response to decitabine treatment and/or Zeb1 knockdown in breast cancer cell lines.

### RNA Sequencing of Breast Tumors

Frozen fragments of breast tumors from patients (BCX PO) and corresponding breast cancer xenografts (BCX P1) were placed in lysis buffer, homogenized, and RNA was isolated using Norgen BIOTEK Total RNA Purification Plus Kit. RNA quality was assessed with an Agilent Bioanalyzer. Genomic RNA was quantified by Picogreen (Invitrogen) and quality was then assessed using 2200 Tapestation (Agilent). RNA was converted to doubled stranded complementary DNA (cDNA) using Ovation RNA-Seq System V2 Kit (Nugen). Libraries of cDNA were made using KAPA kits and capture was performed using Nimblegen whole exome V3 probes. Libraries were then sequenced on a HiSeq 2000 (Illumina) via a version 3 TruSeq paired end flowcell per manufacturer's instructions. RNA and DNA sequencing were paired end 2×100 base pairs. Data analysis was performed using tophat to align paired-end reads to the hg19 version of the reference genome, and htseq-count and bedtools were used to obtain expression counts of genes and exons. Qualities of raw and aligned reads were assessed using FastQC and RSEQC.

### TACTSD2 Gene Expression in Breast Tumors and Cell Lines

To assess expression of TACSTD2 in metaplastic and non-metaplastic breast tumors in TCGA, expression data was downloaded from the cBio Portal “Breast Invasive Carcinoma” Firehouse Legacy data set (https://www.cbioportal.org/). Expression of TACTSD2 was compared between metaplastic and non-metaplastic breast cancer samples. Correlation of TACSTD2 expression in BCX PO (patient) and BCX P1 (xenograft) were assessed. Relative gene expression of TACSTD2 was then compared to expression of EMT regulatory genes in 18 breast tumors (BCX PO) and from breast cancer cell lines within two cancer cell line data repositories: CCLE (sites.broadinstitute.org/ccle/datasets) and LINCS (lincs.hms.harvard.edu/db/datasets/20348/main). Relative expression of TACSTD2 was the compared to expression of CDH1. Analysis was carried out using log 2 normalized counts after removing batch effect, and Spearman correlations were calculated.

### Quantitative RT-PCR

Following treatments, cells were lysed and total RNA was extracted using GeneJet RNA Purification Kit (ThermoFisher) according to manufacturer's manual. cDNA was synthesized from RNA using High-Capacity RNA-to-cDNA Kit (ThermoFisher) following manufacturer's manual. DNA amount in cDNA samples were quantitated using Qubity dsDNA Kit (ThermoFisher). Real time PCR was performed with the same amount DNA from each sample using TaqMan Universal PCR Master Mix Kit (ThermoFisher), under the PCR conditions: 50° C., 2 minutes; 95° C., 10 minutes; [95° C., 15 seconds, 60° C., 1 minutes]×35 cycles. Fold changes of mRNA expression levels were calculated using formula 2\hat{ }rrCT (rrCT: cycle threshold of treatment group-cycle threshold of control group).

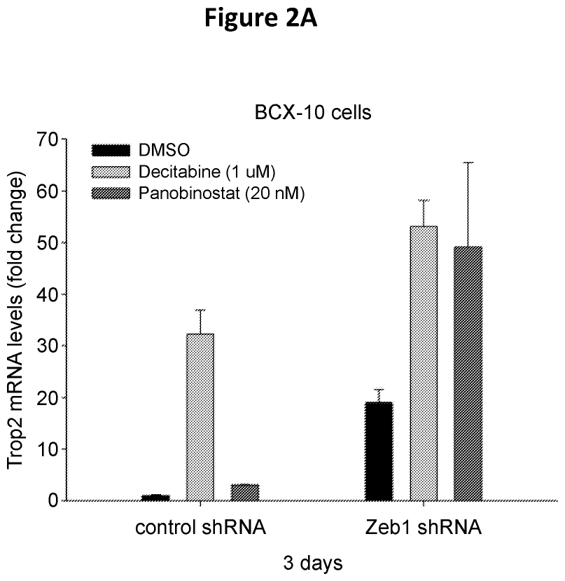

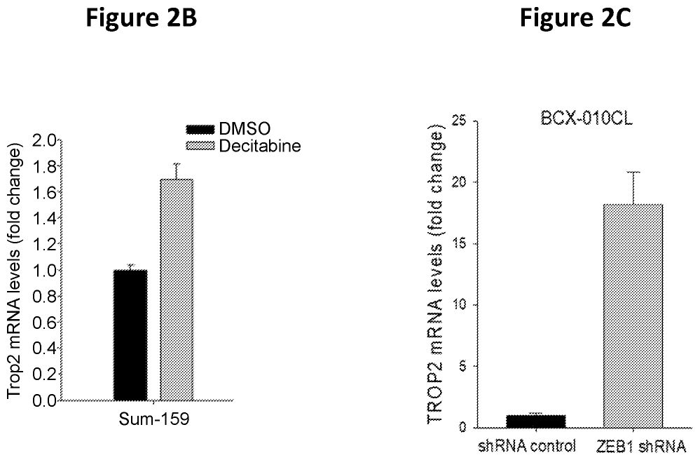

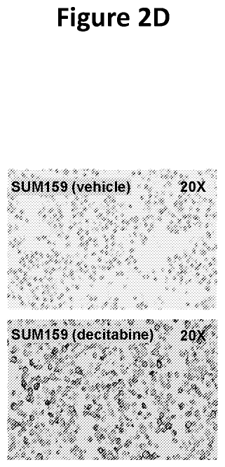

BCX-010 cells (FIG. 2A) and Sum-159 cells (FIG. 2B) were treated decitabine (1 μM), panobinostat (20 nM), or DMSO for three days. Total RNA was extracted from the treated cells using GeneJet RNA kit, followed by reverse transcription using High Capacity RNA-to-cDNA kit. cDNAs were quantitated using Qubit DNA kit. Quantitative real time PCR (qPCR) was performed using Taqman Universal PCR Master Mix kit with a TROP2 probe. Relative TROP2 levels were obtained by normalization with GAPDH. Additionally, BCX-010CL cells were treated with shRNA control or ZEB1 shRNA (FIG. 2C).

After cell fixation, cell pellet blocks were processed and probed with anti-TROP2 antibody, followed by hematoxylin staining (FIG. 2D).

Formalin-fixed paraffin-embedded (FFPE) cell lines/tissue of 4-micron thickness slides were baked at 60° C. for 1 hour. IHC staining was performed using Leica BondRXm autostainer (Leica Biosystems) for further processing. In brief, after deparaffinized by bond dewax solution, epitope retrieval (HIER) was performed with citrate buffer pH6, followed by washing steps. After peroxidase blocking (3% H₂O₂) for 20 minutes, slides were incubated in TROP2 rabbit monoclonal [EPR20043], catalog #ab214488 with a 1:2000 dilution for 15 minutes at ambient temperature. After transient washing, post-primary and polymer incubation for 8 min, followed by hematoxylin staining and wash steps. Afterwards, the slides were unloaded from autostainer, air dried and cover slipped with mounting media (Cytoseal XYL mounting media).

The qPCR results showed that decitabine treatment increased TROP2 mRNA expression in all cell lines with and without Zeb1 shRNA treatment (FIG. 2A-2B). Zeb1 KD treatment increases TROP2 mRNA expression in the transfected BCX-010 cells (FIG. 2A, FIG. 2C). Further, the immunocytochemistry demonstrates that decitabine increases TROP2 expression in SUM159 cells (FIG. 2D).

### Example 3. Decitabine Treatment and Zeb1 KD Decrease DNA Methylation Levels on TROP2 Promoter in BCX-010 Cells

TROP2 promoter methylation was measured in response to decitabine treatment and/or Zeb1 knockdown in breast cancer cell lines.

BCX-010 cells (control and Zeb1 KD cells), were seeded in 6-well plates and treated with decitabine (1 μM) for one and three days. Following treatments, cells were lysed and genomic DNA (gDNA) was extracted using Genomic DNA Purification Kit (ThermoFisher) according to manufacturer's manual. Bisulfite CT conversion of quantitated gDNA was performed using EZ DNA Methylation Kit (Zymo Research) following manufacturer's manual. MSP of TROP2 promoter CpG region was performed using a methylation forward primer, a methylation reverse primer, an un-methylated forward primer, and an un-methylated reverse primer. PCR condition was set up as following: 94° C., 1 minute; [94° C., 30 seconds, 62° C., 30 seconds, 72° C., 45 seconds]×40 cycles; 72° C., 2 minutes, 4° C. About 200 bp PCR product was separated by 2% agarose gel.

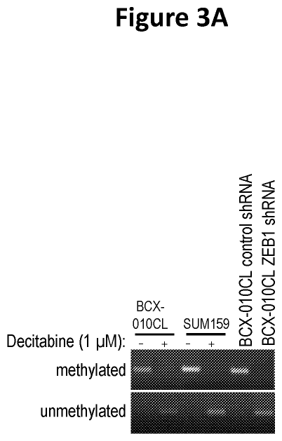

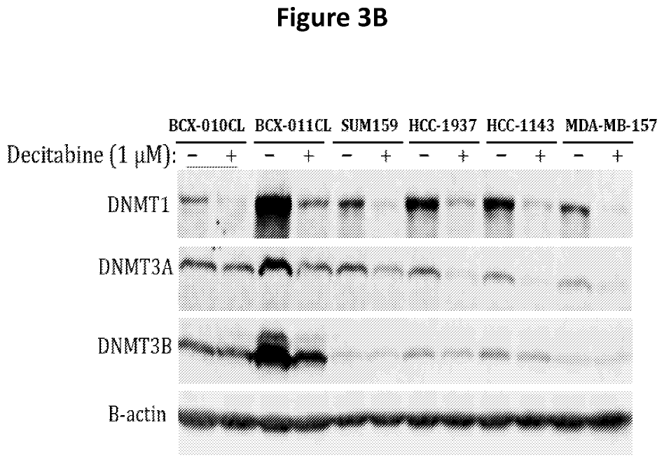

Zeb1 knockdown and decitabine treatment decreased TROP2 promoter methylation both alone and in combination (FIG. 3A). Additionally, immunoblotting of DNA methyltransferases (DNMTs) was performed (FIG. 3B). Breast cancer cell lines were treated with decitabine at 1 μM for 3 days. DNMT isoforms were detected by immunoblotting with antibodies against DNMT1, DNMT3A, and DNMT3B. The data shows that decitabine decreased protein levels of three DNMT isoforms, DNMT1, DNMT3A, and DNMT3B, in the breast cell lines. This effect was also seen in other three breast cancer cell lines that have high TROP2 levels. DNMT1 specific inhibitor GSK-3685032 was also tested. Although GSK-3685032 decreased DNMT1 protein levels, it did not upregulate TROP2 expression in these breast cancer cell lines.

These figures establish that Zeb1 knockdown and/or decitabine treatment act to decrease TROP2 promoter methylation, leading to increased TROP2 expression.

### Example 4. Synergistic Combination Treatment of Decitabine and Sacituzumab Govitecan on Cell Survival in BCX-010 and BCX-011 Cells

In order to determine the effect of decitabine and/or Zeb1 knockdown treatment on sacituzumab govitecan activity, survival assays were performed in breast cancer cell lines.

BCX-010 cells (control and Zeb1 knockdown) and BCX-011 cells were seeded in 96-well plates at densities of 0.15-0.6×10⁴ cells/100 μl per well in triplicates for each treatment dose. After adhering overnight, 100 μl of serially diluted drug solutions (decitabine, or sacituzumab govitecan, or their combination at ratio of 20:1) were added. Cells were incubated at 37° C. for 72 hours. Cells were then fixed with 50% trichloroacetic (TCA) followed by staining with 0.4% sulforhodamine B (SRB) solution. OD values were read at 490 nm by plate reader Synergy 4 (BioTek). The half maximal inhibitory concentration (IC50) was determined using CalcuSyn software (Biosoft). Sigmoid drug-inhibition curves were made using GraphPad Prism v6.05 software. To evaluate combination efficacy, combination index (CI) was determined based on Chou-Talalay IC50 model and isobologram made using CalcuSyn. CI<1.0 (curve left-shift): synergistic; CI=1.0, additive; CI>1.0 (curve right-shift): antagonistic).

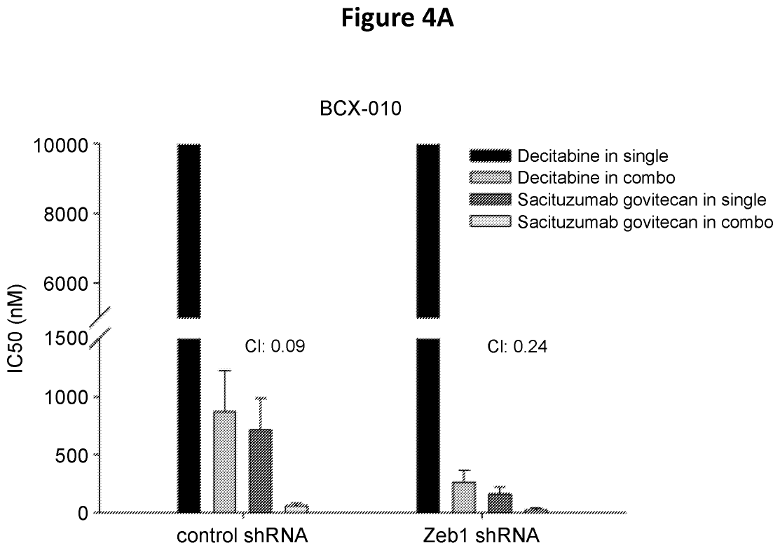

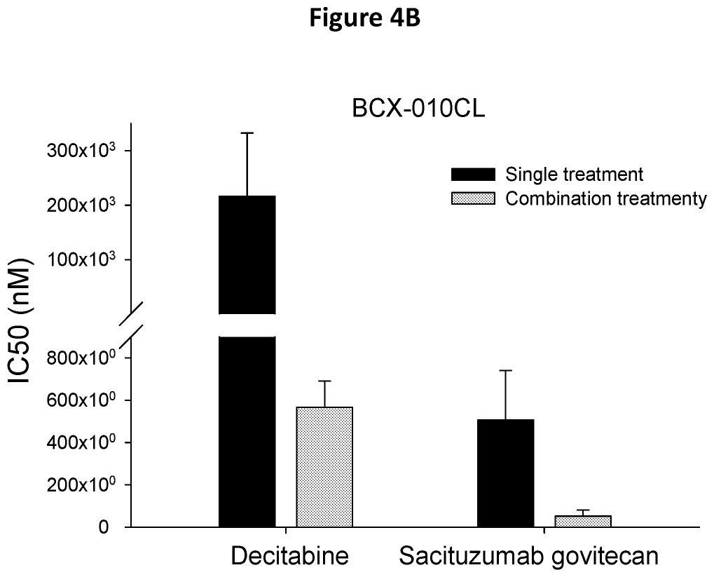

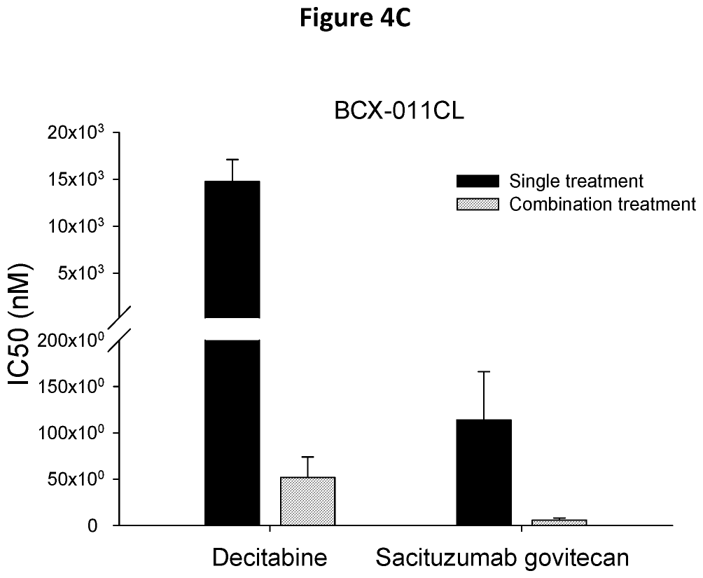

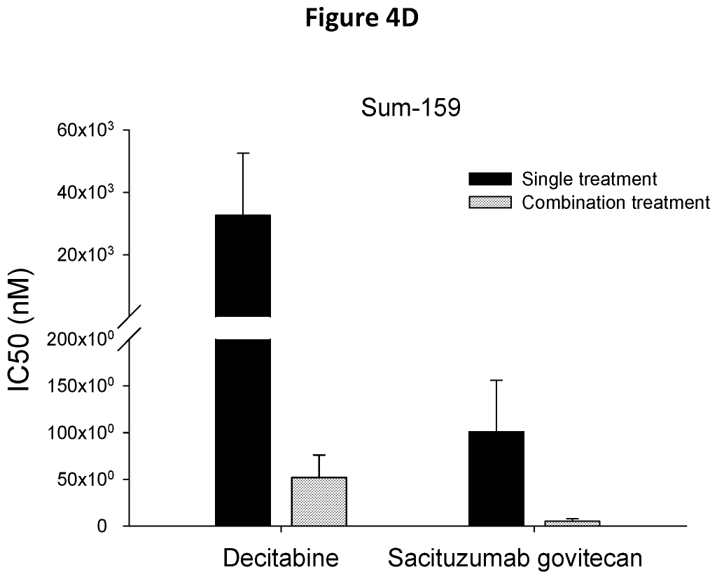

Zeb1 knockdown sensitizes cell response to decitabine and/or sacituzumab govitecan, as shown by decreased IC50s (FIG. 4A). Furthermore, the combination treatment of decitabine with sacituzumab govitecan had synergistic therapeutic efficacy in all three cell lines (FIG. 4A-4D).

### Example 5. Synergistic Combination Treatment of Decitabine with Sacituzumab Govitecan on Apoptosis in BCX-010 and Sum-159 Cells

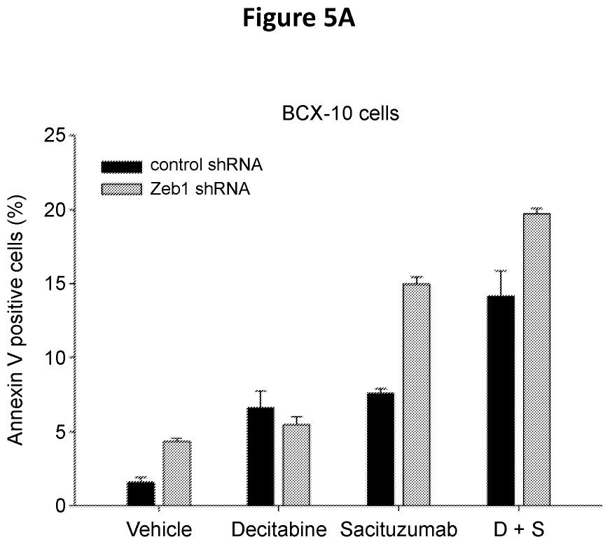

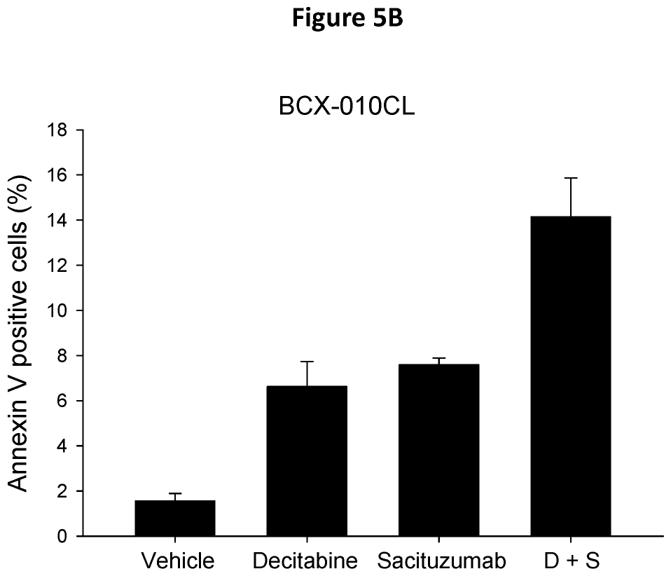

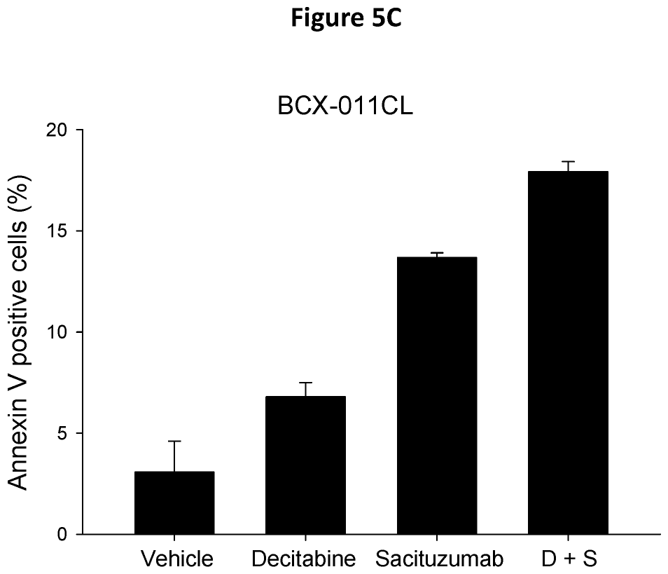

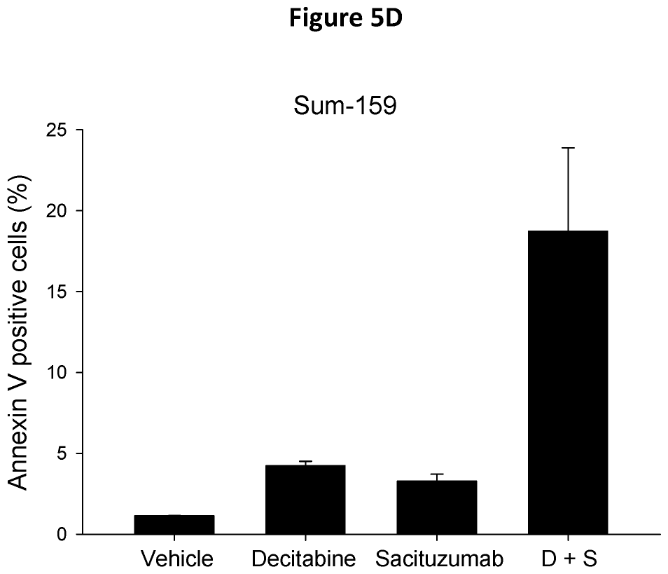

In order to determine the effect of decitabine and/or Zeb1 knockdown treatment on sacituzumab govitecan activity, apoptosis was measured in breast cancer cell lines. BCX-010 cells (FIG. 5A and FIG. 5B), BCX-011 cells (FIG. 5C) and Sum-159 cells (FIG. 5D) were seeded in 6-cm plates, then treated with decitabine (1 μM) and/or sacituzumab govitecan (0.1 μM) for three days in an apoptosis assay. After the three day treatment, cells were stained with Annexin V. Flow cytometry was performed to sort Annexin V positive cells (apoptotic cells) (FIGS. 5A-5D). Percentage of apoptotic cells was calculated by Annexin V positive cells over total cell populations.

Cells were seeded in 6 cm plates at a density of 3×10⁵ cells per well in triplicates for each treatment group. The following day, cells were treated with decitabine and SG at 1 μM and 0.1 μM respectively, or their combination. After 72 hours, floating and attached cells were collected. Using Annexin-V-FLUOS Staining Kit (Roche), cells were stained with Annexin V fluorescence and propidium iodide (PI), following manufacturer's protocol. Samples were analyzed by flow cytometry. Percentage of Annexin V positive apoptotic cells were calculated.

The flow cytometry analysis showed that Zeb1 knockdown induced cell apoptosis in BCX-010 cells (FIG. 5A). Furthermore, decitabine and sacituzumab govitecan alone induced cell apoptosis in BCX-010 and Sum-159 cells (FIGS. 5B-5D). Finally, combination treatment of decitabine with sacituzumab govitecan enhanced induction of apoptosis in all cell lines (FIGS. 5A-5D).

### Example 6. Enhanced Treatment Efficacy with Combination of Decitabine and Sacituzumab Govitecan on Colony Formation in Sum-159 Cells

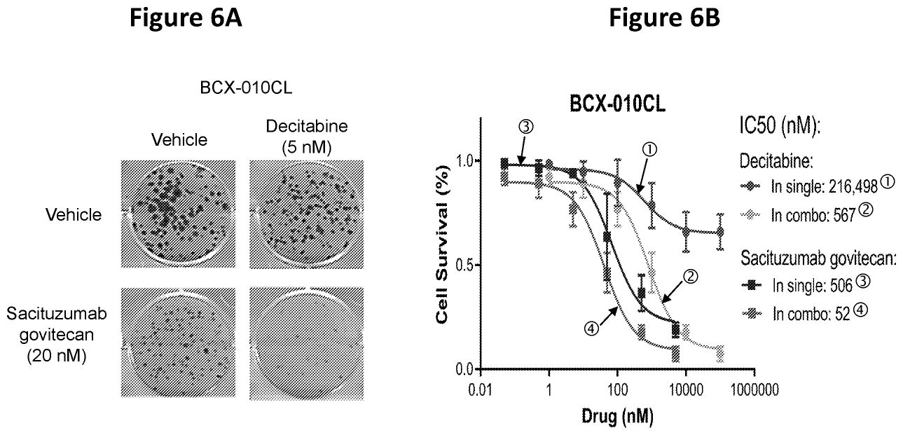

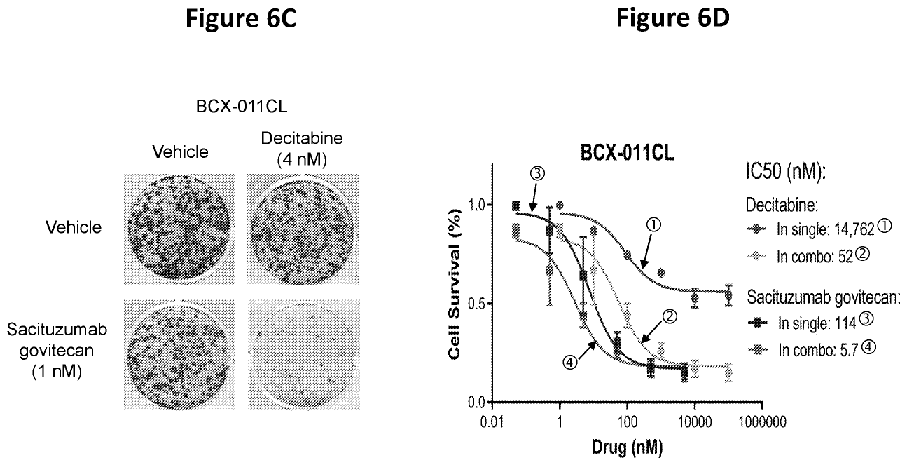

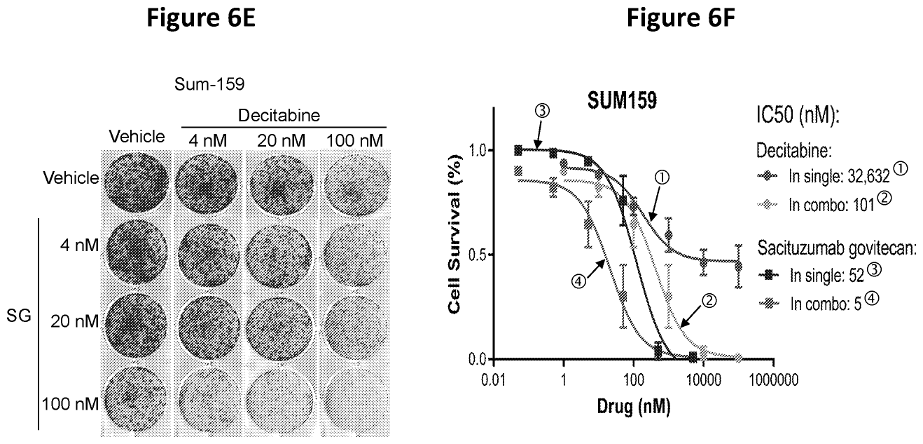

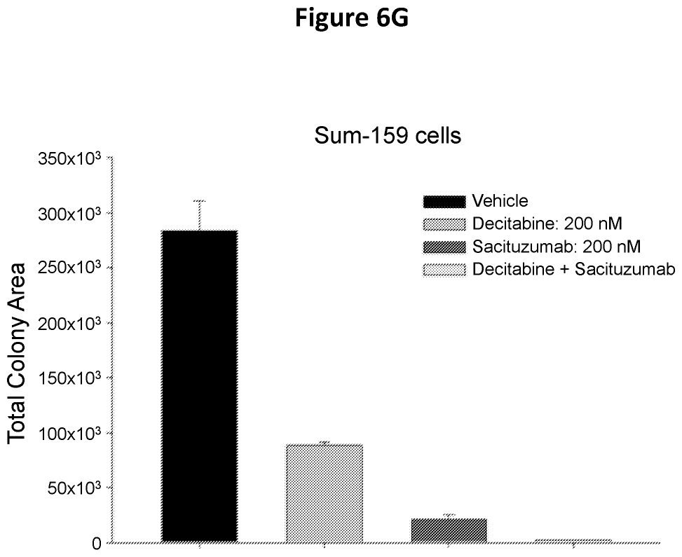

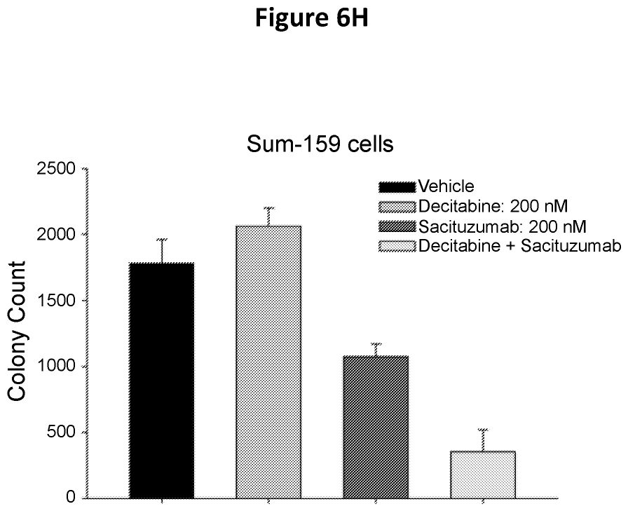

In order to determine the effect of decitabine treatment on sacituzumab govitecan activity, colony formation assays were performed in BCX-010 cells (FIGS. 6A-6B), BCX-011 cells (FIG. 6C-6D), and Sum-159 breast cancer cells (FIG. 6E-6F).

### Colony Formation Assay

Cells were seeded in 6-well plates at a density of 1000 cells per well in triplicates for each treatment group. Next day, cells were treated with single drugs, or combination at the different concentrations. Culture medium was changed with fresh drugs twice a week. Cells were cultured for 3 weeks. Cell colonies were then fixed in 10% formalin and stained with 0.05% crystal violet in 25% methanol. The stained colonies were imaged and total colony area was quantitated using NIH ImageJ v.1.48 software. Effect-based Combination Index (CI) was calculated by inhibition percentage of single drug and combination treatments using Bliss combination model. CI=((EA+EB)−(EA*EB))/EAB, where EA, EB, and EAB are effects of drug A, B and combination AB inhibition percentage. Here the effect is inhibition percentage of colony formation compared to vehicle controls. (CI<1.0: synergistic; CI=1.0: additive; CI>1.0: antagonistic).

Both decitabine and sacituzumab govitecan inhibited cell colony formation in Sum-159 cells. Additionally, the combination treatment of decitabine with sacituzumab govitecan further enhanced the inhibitory effect on colony formation, showing the advantage of using decitabine in combination with sacituzumab govitecan.

### Example 7. Enhanced Treatment Efficacy with Combination of Decitabine and Sacituzumab Govitecan on Colony Formation in BCX-010 Cells

In order to determine the effect of decitabine and/or Zeb1 knockdown treatment on sacituzumab govitecan activity, colony formation assays were performed in BCX-010 breast cancer cells. BCX-010 cells (control and Zeb1 knockdown) were seeded into 6-well plates at 1000 cells/well. Cells were treated with decitabine (5 nM) and/or sacituzumab govitecan (2005 nM) for 14 days. Culture medium with replaced with culture medium containing fresh drugs twice. After 14 days, cell colonies form on the plates were fixed with 10% formalin and stained with 0.05% crystal violet. Total colony area and colony number were quantified using ImageJ software.

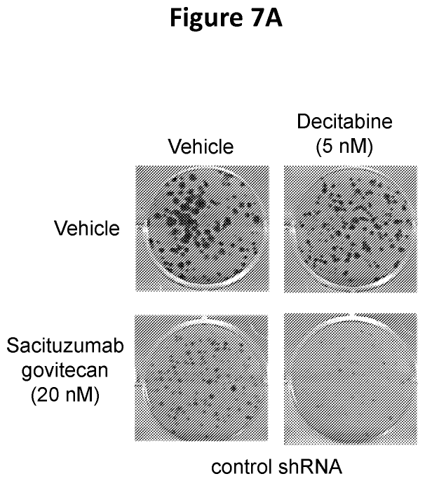

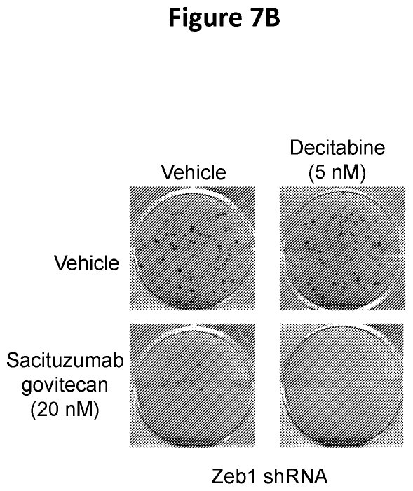

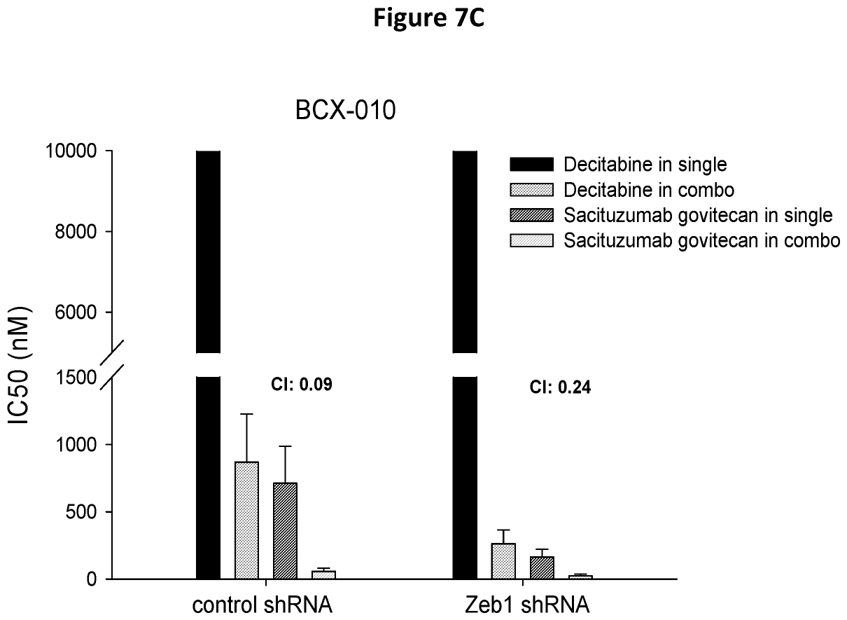

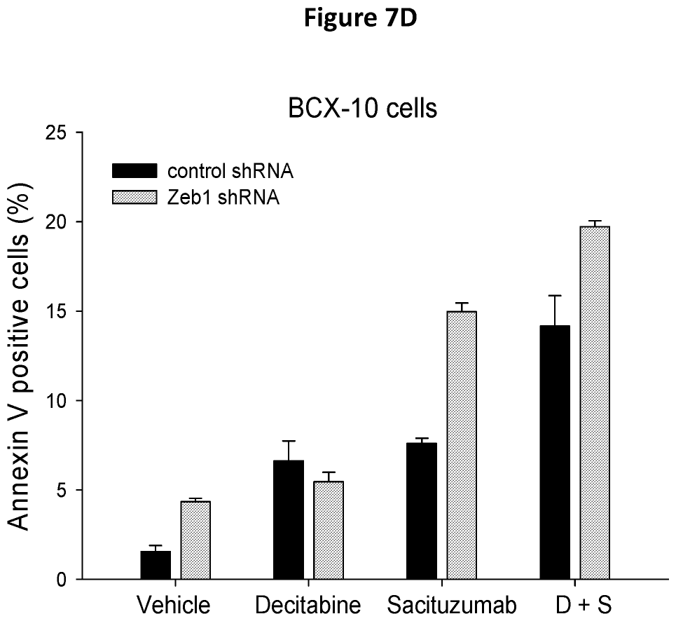

Decitabine and sacituzumab govitecan inhibited cell colony formation in both BCX-010 control (FIG. 7A) and Zeb1 knockdown cell lines (FIG. 7B).

In a cell survival assay, shRNA control and ZEB1 shRNA cell lines were treated with decitabine, sacituzumab govitecan or their combination at concentrations in serial dilutions for 3 days (FIG. 7C). In apoptosis assay, cells were treated with decitabine at 1 μM, SG at 0.1 μM, or their combination for 3 days. Annexin V positive apoptotic cells were measured by flow cytometry. Percentage of apoptotic cells over total cell populations was calculated (FIG. 7D).

The Annexin V staining showed that while ZEB1 deficiency did not change decitabine sensitivity, it significantly raised the percentage of apoptotic cells in both single and combination treatment with SG, compared to control cells. Additionally, combination treatment of decitabine with sacituzumab govitecan further enhanced the inhibitory effect on colony formation, showing the advantage of using decitabine and/or Zeb1 knockdown in combination with sacituzumab govitecan.

### Example 8. Zeb1 Knockdown Reverses EMT in BCX-010 Cells

In order to assess the effect of Zeb1 knockdown treatment on breast cancer cells, endothelial to mesenchymal transition assays were performed in BCX-010 breast cancer cells.

### Cell Migration Assay

Wound healing assay was performed on ZEB1 knockdown cells. When cells cultured in 12-well plates were confluent, a cross scratch was made on the cell layer with 1 ml tip. Medium was changed to remove detached floating cells. Cells were cultured for 2 days to allow cell migration, followed by 10% formalin fixation and crystal violet staining. Photomicrograph of wound gap was performed, followed by quantitation using ImageJ. Relative fraction of wound gap was converted from wound gap area using a formula: wound gap area=(100/% Area)×Total Area.

### Cell Invasion Assay

Transwell invasion assay was performed on Zeb1 knockdown cells. Transwell inserts (Corning, #354578) were coated with basement membrane extract (BME) Matrigel (Corning, #356234). Starved cells were seeded into the inserts at 5×10⁴ cells/insert in 0.3 ml serum-free medium. The inserts were assembled into wells of 24-well plates containing 0.5 ml medium with 10% FBS. Cells were cultured for 24 hours. Cells on the up-surface of the inserts were removed by scrubbing with cotton swabs. Cells that invaded through the BME were fixed on the down-surface of the inserts with 10% formalin, followed by staining with 0.4% crystal violet. The stained cells were imaged by inverted microscope at ×100 magnifications. Cells numbers were counted per image field. Nine fields were quantitated for each group.

### Anchorage-Independent Cell Growth Assay

Soft agar assay was performed on ZEB1 knockdown cells. 6-well plates were coated with 0.5% bottom agar (Difco Agar Noble, BD, #214220). Cells were mixed with 0.35% top agar containing 10% FBS and seeded onto the bottom agar wells at 5000 cells/well. 1 ml complete medium was added on top the agar. Cells were fed twice a week and cultured for 3 weeks. Colonies in the agar were stained with iodonitrotetrazolium chloride (INT) overnight, followed by colony photomicrograph.

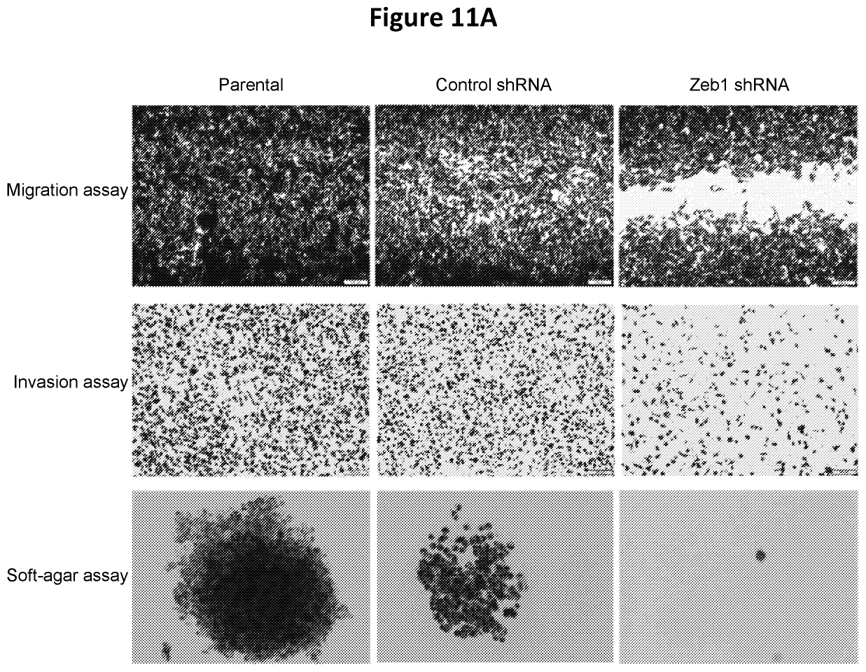

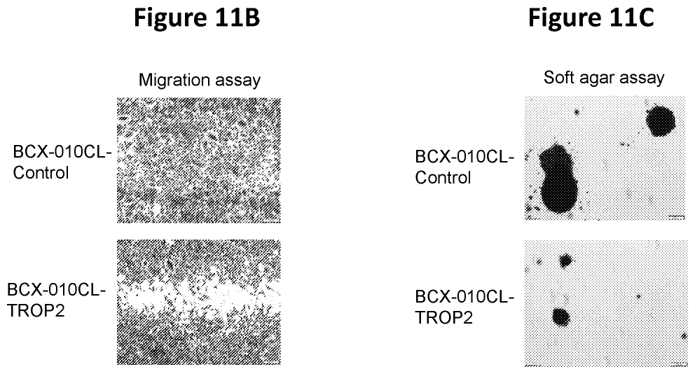

The migration assay showed that Zeb1 knockdown inhibited cell migration from the edges of scratch (FIG. 11A, top row). The invasion assay showed that Zeb1 knockdown inhibited cell invasion through Matrigel (FIG. 11A, middle row). Finally, the soft agar assay showed that Zeb1 knockdown inhibited cell anchorage-independent growth (FIG. 11A, bottom row).

### Example 9. TROP2 Overexpression Increases Cell Sensitivity to Sacituzumab Govitecan in Breast Cancer Cell Lines

In order to assess the effect of TROP2 overexpression on breast cancer cell line sensitivity to Sacituzumab Govitecan, BCX-010CL cells were transfected with TROP2 expression plasmid or empty vector.

### Overexpression of TROP2

For TROP2 overexpression, breast cancer cells seeded in 6-well plates were transfected with TROP2 expression plasmid pCMV6-entry-TROP2 using Lipofectamine 3000 Kit. Two days after transfection, cells were treated with G-418 for 2-3 passages.

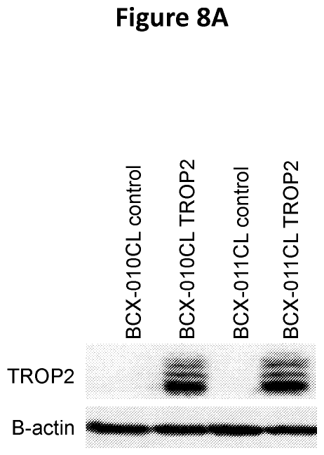

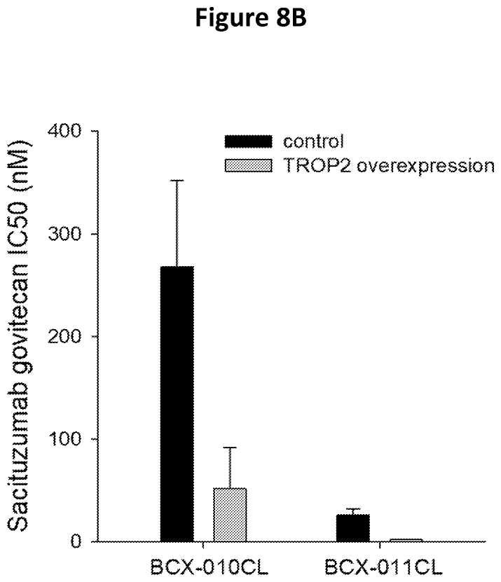

After G-418 selection, immunoblotting was performed to detect TROP2 expression (FIG. 8A). Next, TROP2-overexpressing cells and control cells were treated with SG at concentrations in serial dilutions for 3 days. SG IC50 was calculated (FIG. 8B). Overexpression of TROP2 sensitizes cells to Sacituzumab govitecan in BCX-010CL and BCX-011CL cells (cell survival assay-FIG. 8B).

Further, an Epithelial-mesenchymal transition assays on BCX-010 cell lines transfected with TROP2 or control was performed. Migration assays (FIG. 11B) and a soft agar assay (analyzing anchorage-independent growth) (FIG. 11C) show that overexpression of TROP2 reverses EMT in breast cancer cells.

### Immunofluorescence of ADC Binding on Cell Membrane

ADC drug SG was labeled with alexa fluorescent dye using Alexa Fluo 488 Conjugation Lightening Kit (Abcam, #ab236553), following the manufactory protocol. Final SG-Alexa-488 concentration was 1 μg/μl. To bind ADC to cells, cells were seeded on cover slices in 6-well plates overnight, followed by blocking with 3% BSA/PBS at room temperature for 1 hour. Then cells were incubated with SG-Alexa-488 at 2 μg/ml at room temperature for 2 hours, followed by PBS wash twice. For internalization assay, SG-Alexa-488 was added into the culture medium in the wells at 2 μg/ml for 10 min, 3 hours, and 24 hours. Cells were fixed with 4% paraformaldehyde at room temperature for 15 min, PBS wash twice. The cover slices were mounted onto slide with DAPI counterstaining. Fluorescence on cell surface and in cytoplasm was monitored using Olympus BX51 fluorescent microscope and imaged using Nuance imaging system.

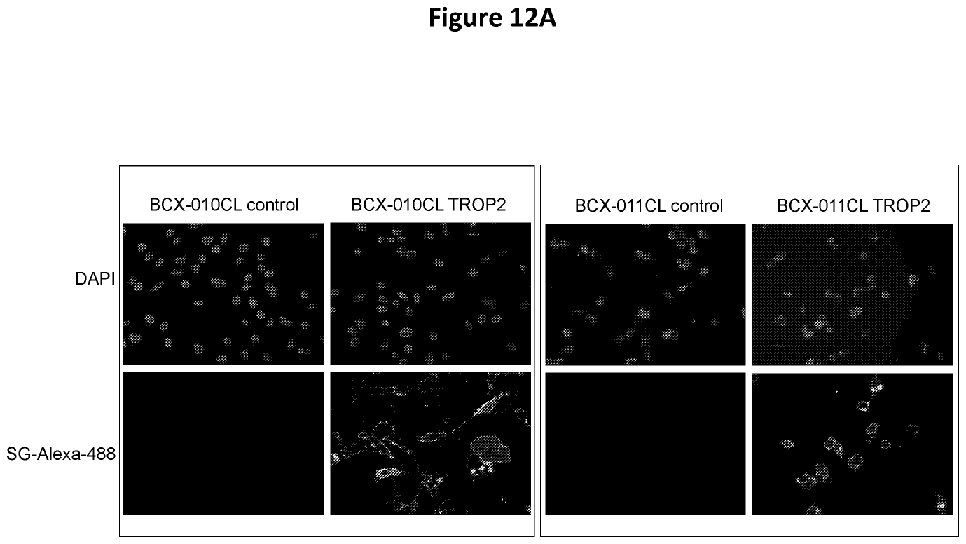

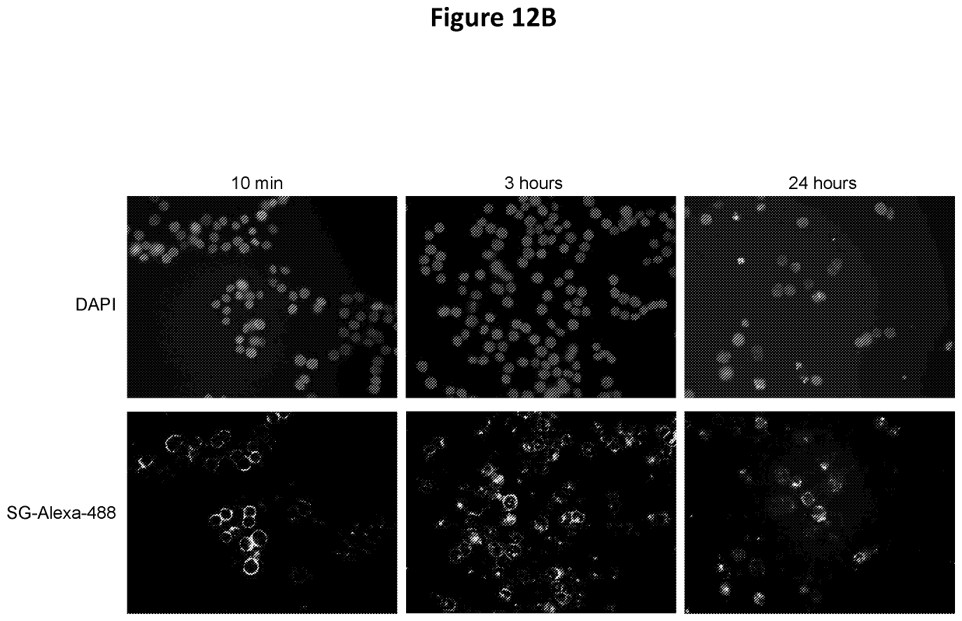

Next, Immunofluorescence assay showed that when these BCX cells were incubated with SG ADC labeled with Alexa-Fluo-488, TROP2-overexpressing cells displayed strong immunofluorescent signal on cell membrane, compared to negative signal in control cells (FIG. 12A). The immunofluorescent signal intensity in these TROP2-overexpressing cells was similar to that in MDA-MB-468 cells that express high levels of endogenous TROP2 (FIG. 12B).

### Example 10. Combination of Decitabine and Sacituzumab Govitecan Effect on Signaling Activity in DDR and Apoptotic Pathways in Breast Cancer Cell Lines

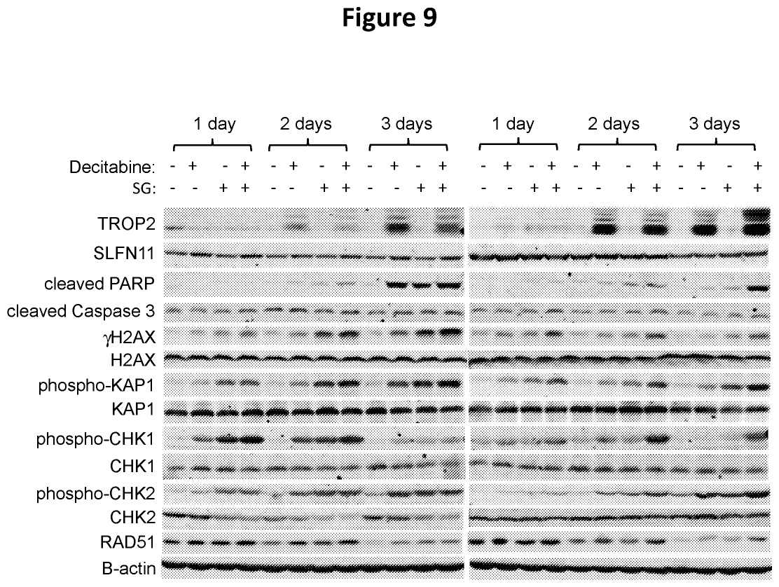

In order to assess the effect of decitabine and Sacituzumab govitecan on cell signaling, breast cancer cell lines were analyzed. BCX-010CL (left panel) and SUM159 (right panel) cells were treated with decitabine at 1 μM, sacituzumab govitecan at 0.1 μM, and their combination for 1, 2, 3 days. Cell lysates were subjected to SDS-PAGE, followed by immunoblotting using antibodies against cleaved PARP1, cleaved Caspase 3, TROP2, SLFN11, and RAD51, and also against phospho-KAP1, γH2AX, phospho-CHK1, phospho-CHK2, and their total proteins. β-actin served as protein loading control (FIG. 9). Ultimately, the combination of decitabine and Sacituzumab govitecan enhances signaling activity in DDR and apoptotic pathways in breast cancer cell lines. By targeting TROP2, the combinatorial treatment of SG and decitabine activated the DDR pathway, as evidenced by elevated phosphoprotein levels of H2AX, CHK1, CHK2, and KAP1 compared to single agent treatment. Thus, the data showed that combination treatment with decitabine and SG significantly enhanced their apoptotic efficacy compared to single agent treatment, as evidenced by Annexin V staining and increased cleaved PARP and caspase 3.

### Example 11. Upregulation of SFN11 Expression Sensitizes Cells to Sacituzumab Govitecan and SN-38 Payload

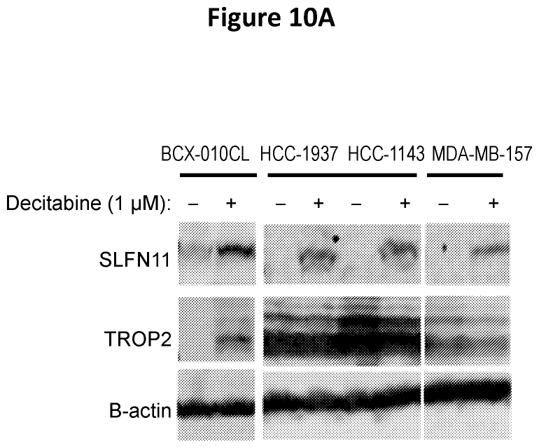

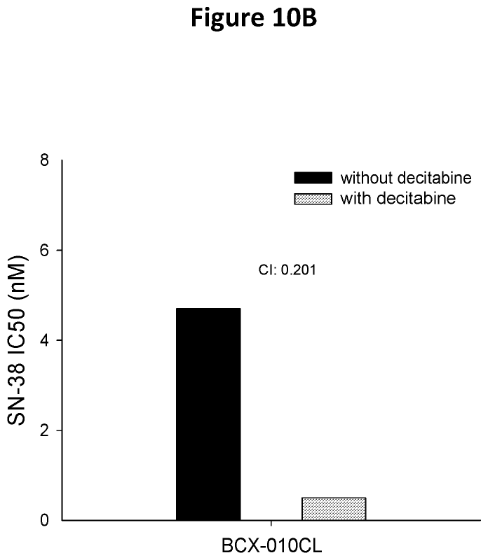

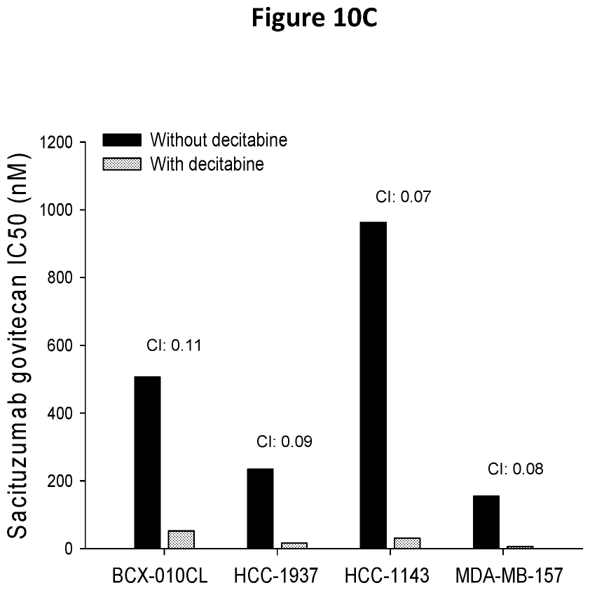

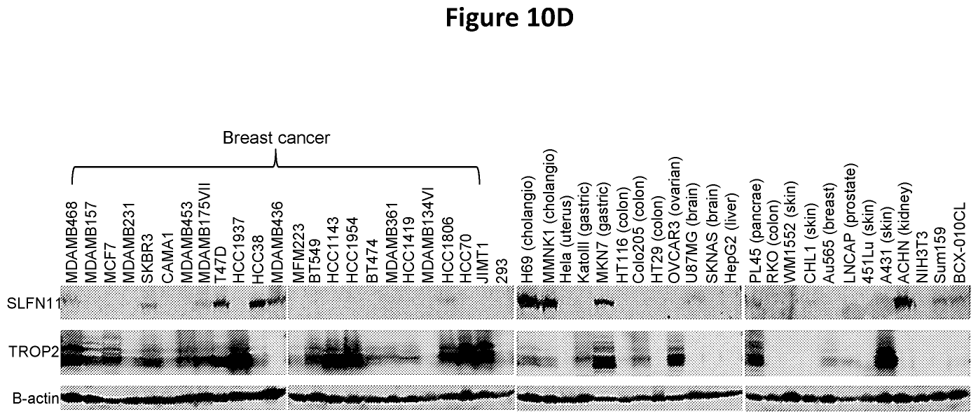

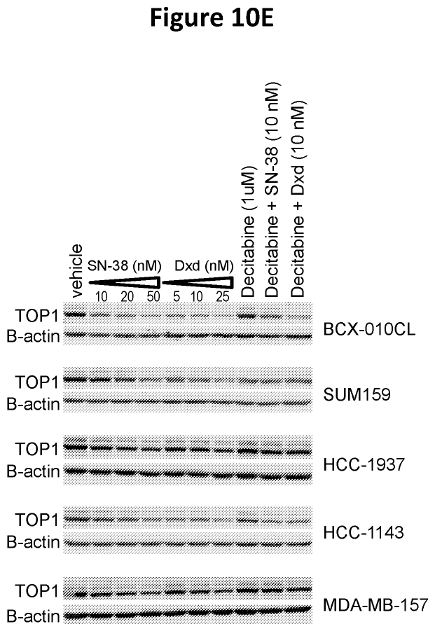

SLFN11 and TROP2 protein expression in a panel of 48 cell lines including 27 breast cancer cell lines was screened by immunoblotting (FIG. 10D). Additionally, cell lines BCX-010CL, HCC-1937, HCC-1143, and MDA-MB-157 were treated with decitabine at 1 μM for 3 days. SLFN11 and TROP2 proteins in cell lysates were detected by immunoblotting. Decitabine increases SLFN11 expression which sensitizes cells to Sacituzumab Govitecan and its payload SN-38 in breast cancer cell lines (FIG. 10A). BCX-010CL cells were also treated with SN-38 at serial dilutions, with or without decitabine co-treatment for 3 days. IC50s of individual agents and combination index were calculated. This cell survival assay showed that decitabine sensitizes cells to SN-38 in BCX-010CL cells (FIG. 10B). Finally, BCX-010CL, HCC-1954, HCC-1143, and MDA-MB-157 cells were treated with Sacituzumab govitecan at serial dilutions, with or without decitabine co-treatment for 3 days. IC50s of individual agents and combination index were calculated. This cell survival assay showed that decitabine sensitizes cells to Sacituzumab govitecan in breast cancer cell lines (FIG. 10C). TOP1 inhibitors, such as irinotecan and its active metabolite SN-38, are chemotherapy agents which cause double strand DNA damage. In addition to inhibiting TOP1 activity, the data and other studies demonstrated that TOPO I inhibitors also reduce expression of the enzyme FIG. 10E).

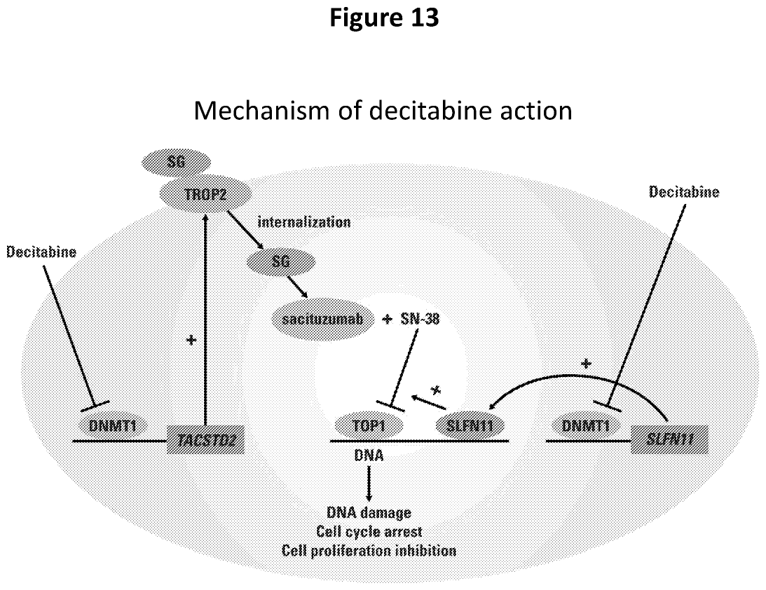

This data shows that in tumor cells, decitabine decreases promoter methylation of the TACSTD2 gene by inhibiting DNA methyltransferase 1 (DNMT1), activating expression of TACSTD2/TROP2. On cell membrane, TROP2 protein binds to TROP2 ADC drug sacituzumab govitecan (SG). After internalization, SG is proteolytically cleaved in lysosome to release payload SN-38. SN-38 then moves into nucleus where it inhibits topoisomerase 1 (TOP1), leading to DNA damage, cell cycle arrest, and cell death. Decitabine also increases SLFN11 expression which further strengthens the chemocytoxic effect of SN-38 in the tumor cells (FIG. 13). Therefore, the synergistic combination therapy of SG with decitabine could be explained by two separate pharmacological mechanisms: (1) decitabine increases TROP2 expression which is the target of TROP2 ADC sacituzumab govitecan; (2) decitabine also increases SLFN11 expression which sensitizes cells to TOP1 inhibitor payload SN-38.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

1. A method of treating a tumor or a cancer in a subject in need thereof comprising administering to the subject an anti-TROP2 antibody drug conjugate (ADC) and a therapeutic agent that increases TROP2 expression.

2. The method of claim 1, wherein the therapeutic agent comprises a DNA methyltransferase inhibitor.

3. The method of claim 2, wherein the DNA methyltransferase inhibitor is decitabine.

4. The method of claim 1, wherein the therapeutic agent comprises a Zinc Finger E-Box Binding Homeobox 1 (Zeb1) inhibitor.

5. A method of treating a tumor or a cancer in a subject in need thereof comprising administering to the subject an anti-TROP2 antibody drug conjugate (ADC) and a therapeutic agent comprising a DNA methyltransferase inhibitor and/or a Zinc Finger E-Box Binding Homeobox 1 (Zeb1) inhibitor.

6. The method of claim 4, wherein the Zeb1 inhibitor is selected from the group consisting of a short interfering RNA (siRNA), a short hairpin RNA (shRNA), micro RNA (miRNA) or an antisense nucleic acid molecule.

7. (canceled)

8. The method of claim 1, wherein the anti-TROP2 antibody drug conjugate comprises a cytotoxic drug selected from the group consisting of an anthracycline, a camptothecin, a tubulin inhibitor, a maytansinoid, a calicheamycin, an auristatin, a nitrogen mustard, an ethylenimine derivative, an alkyl sulfonate, a nitrosourea, a triazene, a folic acid analog, a taxane, a COX-2 inhibitor, a pyrimidine analog, a purine analog, an antibiotic, an enzyme inhibitor, an epipodophyllotoxin, a platinum coordination complex, a vinca alkaloid, a substituted urea, a methyl hydrazine derivative, an adrenocortical suppressant, a hormone antagonist, an antimetabolite, an alkylating agent, an antimitotic, an anti-angiogenic agent, a tyrosine kinase inhibitor, an mTOR inhibitor, a heat shock protein (HSP90) inhibitor, a proteosome inhibitor, an HDAC inhibitor, a topoisomerase inhibitor and a pro-apoptotic agent.

9. The method of claim 1, wherein the anti-TROP2 antibody drug conjugate comprises a cytotoxic drug selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, COX-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), a pro-drug form of 2-PDox (pro-2-PDox), cyano-morpholino doxorubicin, doxorubicin glucuronide, endostatin, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosurea, olaparib, plicomycin, procarbazine, paclitaxel, PCI-32765, pentostatin, PSI-341, raloxifene, semustine, SN-38, sorafenib, streptozocin, SU11248, sunitinib, tamoxifen, temazolomide, transplatinum, thalidomide, thioguanine, thiotepa, teniposide, topotecan, uracil mustard, vatalanib, vinorelbine, vinblastine, vincristine, vinca alkaloids and ZD1839.

10. (canceled)

11. The method of claim 1, wherein the anti-TROP2 antibody drug conjugate comprises a SN-38 cytotoxic drug.

12. The method of claim 1, wherein the anti-TROP2 antibody drug conjugate comprises sacituzumab or a functional fragment thereof, or datopotamab or a functional fragment thereof.

13. The method of claim 1, wherein the anti-TROP2 antibody drug conjugate is selected from the group consisting of sacituzumab govitecan, datopotamab deruxtecan, and SKB264.

14. The method of claim 1, wherein the cancer is carcinoma.

15. The method of claim 1, wherein the cancer is selected from the group consisting of brain cancer, breast cancer, cervical cancer, colorectal cancer, esophageal cancer, lung cancer, lymphomas, ovarian cancer, pancreatic cancer, prostate cancer, stomach cancer, thyroid cancer, bladder cancer, and uterine cancer.

16. The method of claim 15, wherein the breast cancer is triple negative breast cancer.

17. The method of claim 15, wherein the breast cancer is HER2 negative breast cancer.

18. The method of claim 15, wherein the bladder cancer is urothelial cancer.

19. The method of claim 15, wherein the uterine cancer is endometrial cancer.

20.-26. (canceled)

27. A method of treating a subject in need thereof comprising:
(i) determining TROP2 expression level in a tumor sample from the subject;
(ii) administering to the subject having a low expression level of TROP2 in the tumor relative to a reference sample an anti-TROP2 antibody drug conjugate and a therapeutic agent that increases expression of TROP2.

28.-55. (canceled)

56. A composition comprising an anti-TROP2 antibody drug conjugate and a therapeutic agent that increases TROP2 expression.

57.-63. (canceled)

64. A kit for treating a subject in need thereof comprising (a) anti-TROP2 antibody drug conjugate and (b) a therapeutic agent that increases TROP2 expression.

65.-77. (canceled)

78. A combination therapy for the treatment of cancer in a subject, wherein the combination therapy comprises an anti-TROP2 antibody drug conjugate and a therapeutic agent that increases TROP2 expression.

79.-94. (canceled)
